(19) United States
(12) Patent Application Publication
(10) Pub. No.: US 2018/0294047 A1
(43) Pub. Date: Oct. 11, 2018

(54) PERSONAL DATA MARKETPLACE FOR GENETIC, FITNESS, AND MEDICAL INFORMATION INCLUDING HEALTH TRUST MANAGEMENT

(71) Applicant: Seqster PDM, Inc., San Diego, CA (US)

(72) Inventors: Dana HOSSEINI, San Diego, CA (US); Ardavan ARIANPOUR, San Diego, CA (US); Xiang LI, San Diego, CA (US)

(21) Appl. No.: 15/908,477

(22) Filed: Feb. 28, 2018

Related U.S. Application Data
(60) Provisional application No. 62/465,664, filed on Mar. 1, 2017.

(51) Int. Cl. G16H 10/60; G06F 17/30

(52) U.S. Cl. CPC G16H 10/60; G06F 17/30569

(57) ABSTRACT

Disclosed herein are methods, systems, and media for managing health information that enable individuals to create a profile, associate fitness data, genetic data, and medical data and designate one or more recipient successors for all or part of their profile to create a health trust. Also disclosed herein are methods, systems, and media for providing a marketplace for health information by maintaining a database of profiles, each profile including personal information, fitness data, genetic data, and medical data for the individual, applying an algorithm to generate a monetary market value for each profile, enabling health data consumers to conduct a search of the database, and enabling the health data consumers to select identified profiles and subscribe to the selected profiles.

### CROSS-REFERENCE TO RELATED APPLICATIONS

This application claims the benefit of U.S. Provisional Application Ser. No. 62/465,664 filed Mar. 1, 2017, the entire contents of which are hereby incorporated by reference.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### BACKGROUND OF THE INVENTION

Genetic information pertaining to individuals is increasingly prevalent. Since the completion of the Human Genome Project, technological improvements and automation have increased speed and lowered costs to the point where individual genes and whole genomes can be sequenced routinely. An entire genome can be sequenced for just a few thousand dollars and that price is projected to fall to less than a hundred dollars in a few years thus making the technology accessible to a broader market. However, despite this, very few individuals have had any genetic sequencing performed. Moreover, spurred in part by recent legislation, use of electronic medical records, systematized collections of digital patient health information, is on the rise. Finally, personal fitness tracking devices are nearly ubiquitous and round out available health data by generating vast troves of fitness data for individuals.

### SUMMARY OF THE INVENTION

While large quantities of personal health data, including genetic, medical (e.g., electronic and/or paper health records), and fitness data are available in various forms, from various sources, existing technologies do not adequately aggregate, organize, and store this data to unlock its potential. Together, genetic, medical, and fitness data create a robust health picture of an individual and can provide actionable intelligence regarding one's health. However, current technologies fail to bring genetic, medical, and fitness data together in a way that is accessible and convenient for consumers.

Additionally, existing services do not acknowledge the true market value of properly aggregated health data for individuals. As such, current technologies do not calculate the value of such data, do not create efficient and fluid marketplaces around these valuable assets, do not provide any mechanisms for increasing the value of each individual's health data and do not enable individuals to realize health data as a new asset class. Importantly, no tools currently exist to allow individuals to pass their valuable health information to their successors and have the entrusted health data add considerable value to the recipients' own health data similar to other assets. In addition, existing technologies do not provide adequate privacy for individual's health data, do not allow an individual to exert sufficient control over who their health data is shared with, what is shared, and the terms of any sharing.

In contrast, the platforms, systems, media, and methods disclosed herein conveniently ingest, aggregate, organize, and store health data including, but not limited to personal data, genetic data, epigenetic data, metabolic data, proteomic data, microbiomic data, electronic and/or paper health records, medical data, laboratory results, and fitness data for individuals. This health data combines synergistically to create a valuable health profiles. The subject matter described herein provides tools allowing individuals to update and expand their health data profile to increase its value. Moreover, provided herein are easy-to-use tools that allow individuals to create a health trust that not only adds to the value of their data, but safeguards it by providing access to their designated successors. This is done while safeguarding the privacy of each individual's health data by allowing individuals to control what is shared, to whom, and under what terms and conditions.

In addition, the platforms, systems, media, and methods disclosed herein create a marketplace around this health data. In some cases, the marketplace is combined with machine learning algorithms and/or manual curation to recommend the most salient data to data consumers and make that data available for the data consumers to subscribe to and/or license, or alternatively, allow data consumers to search, filter, sort, and browse profiles. In this marketplace, data consumers subscribe to profiles of interest and individuals monetize their personal health data. In combination, the disclosed health trusts and the health data marketplace allow families to create valuable pools of health data, keep the data updated, and monetize their collective data into the futures.

In one aspect, disclosed herein are computer-implemented methods of securely managing health information comprising: providing tools for an individual to create a profile, the profile comprising personal information; ingesting fitness data, genetic data, and medical data for the individual and associating the fitness data, the genetic data, and the medical data with the profile; and providing tools for the individual to designate one or more recipient successors for all or part of the profile and the associated data to create a health trust. In some cases, the computer-implemented methods of securely managing health information include harmonizing the ingested data through a staged transformation process while maintaining integrity and provenance of the data, the staged transformation process comprising: parsing the ingested data according to source type; and standardizing the parsed data into a consistent set of terms; thereby forming a health data interoperability framework. The method may further include formatting the data into JavaScript Object Notation (JSON) format and storing the formatted data in structured relational database tables. The method may also include providing an ontology mapping and guided curation tool to enable medical staff to standardize any medical term into the health data interoperability framework. The computer-implemented methods of securely managing health information, in some cases, include providing tools for recognizing ownership of health information and providing tools for the individual to manage privacy settings for themselves and/or their health information. Optionally, the individual designates each recipient as a successor for one or more of: the personal information, the fitness data, the genetic data, and the medical data, e.g., electronic health records. The individual may designate each recipient successor by inputting a name, a relationship to the individual, and contact information for the recipient successor. In some cases, the method further comprises sending an invitation to each designated recipient successor, the invitation comprising a feature allowing the recipient to accept the invitation. In such cases, the one or more recipient successors accepting the invitation have access to all or part of the profile and the associated data. The personal information may comprise social networking information and may comprise family tree information. In some cases, the fitness data comprises data generated by a fitness tracking device or input by the user into a fitness log. In such cases, the fitness data may be ingested by accessing an API or by receiving upload of one or more data files provided by the individual. In some cases, the genetic data comprises nucleic acid sequence information. In such cases, the nucleic acid sequence information comprises DNA sequence information or RNA sequence information. The genetic data may be ingested by accessing an API or by receiving upload of one or more data files provided by the individual. In some cases, the medical data comprises at least one electronic health record (EHR) or at least one personal health record (PHR). In such case, the medical data may be ingested by accessing an API or by receiving upload of one or more data files provided by the individual. The method may, in some cases, further comprise providing a coupon, discount, or subsidy for genetic sequencing services in response to the ingesting of the fitness data, the medical data, or both the fitness data and the medical data. The method, in some cases, further comprises providing a coupon, discount, or subsidy for health care services. Also, the method may further comprise generating and presenting a genetic data summary for the individual comprising a plurality of phenotypes, the genetic data summary further comprising at least one annotation, at least one article, and a confidence level associated with each phenotype. In some cases, the method further comprises applying an algorithm to generate a monetary market value for the profile and the associated data. In such cases, the monetary market value may be based, at least in part, on one or more of: the quantity of the data, the quality of the data, the number of types of data, the number of family members of the individual who have profiles, and whether a health trust is established.

In another aspect, disclosed herein are computer-implemented systems comprising: a digital processing device comprising: at least one processor, an operating system configured to perform executable instructions, a memory, and a computer program including instructions executable by the digital processing device to create a health dashboard application comprising: a software module presenting an interface for creating a profile for an individual, the profile comprising personal information; a software module ingesting fitness data, genetic data, and medical data for the individual and associating the fitness data, the genetic data, and the medical data with the profile; and a software module presenting an interface for allowing the individual to designate one or more recipient successors for all or part of the profile and the associated data to create a health trust. In some cases, the health dashboard application includes a software module harmonizing the ingested data through a staged transformation process while maintaining integrity and provenance of the data, the staged transformation process comprising: parsing the ingested data according to source type; and standardizing the parsed data into a consistent set of terms; thereby forming a health data interoperability framework. The health dashboard application may include a software module formatting the data into JavaScript Object Notation (JSON) format and storing the formatted data in structured relational database tables. The health dashboard application may also provide an ontology mapping and guided curation tool to enable medical staff to standardize any medical term into the health data interoperability framework. In some cases, the interface for allowing the individual to designate one or more recipient successors optionally comprises features allowing the individual to designate each recipient as a successor for one or more of: the personal information, the fitness data, the genetic data, and the medical data. The interface for allowing the individual to designate one or more recipient successors may, in some cases, comprise features allowing the individual to designate each recipient by inputting a name, a relationship to the individual, and contact information for the recipient. In some cases, the application further comprises a software module sending an invitation to each designated recipient successor, the invitation comprising a feature allowing the recipient to accept the invitation. In such cases, the one or more recipient successors accepting the invitation have access to all or part of the profile and the associated data. The personal information optionally comprises social networking information and optionally comprises family tree information. In some cases, the fitness data comprises data generated by a fitness tracking device or input by the user into a fitness log. In such cases, the fitness data may be ingested by accessing an API or by upload of one or more data files provided by the individual. In some cases, the genetic data comprises nucleic acid sequence information. In such cases, the nucleic acid sequence information comprises DNA sequence information or RNA sequence information. The genetic data may be ingested by accessing an API or by upload of one or more data files provided by the individual. In some cases, the medical data comprises at least one electronic health record (EHR) or at least one personal health record (PHR). In such cases, the medical data is ingested by accessing an API or by upload of one or more data files provided by the individual. The application may further comprise a software module providing a coupon, discount, or subsidy for genetic sequencing services in response to the ingesting of the fitness data, the medical data, or both the fitness data and the medical data. Also, the application may further comprise a software module generating and presenting a genetic data summary for the individual, the genetic data summary comprising a plurality of phenotypes and further comprising at least one annotation, at least one article, and a confidence level associated with each phenotype. In some cases, the application further comprises a software module applying an algorithm to generate a monetary market value for the profile and the associated data. In such cases, the monetary market value is based, at least in part, on one or more of: the quantity of the data, the quality of the data, the number of types of data, the number of family members of the individual who have profiles, and whether a health trust is established. The application is optionally implemented as software-as-a-service (SaaS).

In another aspect, disclosed herein are non-transitory computer-readable storage media encoded with a computer program including instructions executable by a processor to create a health dashboard application comprising: a software module presenting an interface for creating a profile for an individual, the profile comprising personal information; a software module ingesting fitness data, genetic data, and medical data for the individual and associating the fitness data, the genetic data, and the medical data with the profile; and a software module presenting an interface for allowing the individual to designate one or more recipient successors for all or part of the profile and the associated data to create a health trust. In some cases, the health dashboard application includes a software module harmonizing the ingested data through a staged transformation process while maintaining integrity and provenance of the data, the staged transformation process comprising: parsing the ingested data according to source type; and standardizing the parsed data into a consistent set of terms; thereby forming a health data interoperability framework. The health dashboard application may include a software module formatting the data into JavaScript Object Notation (JSON) format and storing the formatted data in structured relational database tables. The health dashboard application may also provide an ontology mapping and guided curation tool to enable medical staff to standardize any medical term into the health data interoperability framework. In some cases, the interface for allowing the individual to designate one or more recipient successors optionally comprises features allowing the individual to designate each recipient as a successor for one or more of: the personal information, the fitness data, the genetic data, and the medical data. The interface for allowing the individual to designate one or more recipient successors may, in some cases, comprise features allowing the individual to designate each recipient by inputting a name, a relationship to the individual, and contact information for the recipient. In some cases, the application further comprises a software module sending an invitation to each designated recipient successor, the invitation comprising a feature allowing the recipient to accept the invitation. In such cases, the one or more recipient successors accepting the invitation have access to all or part of the profile and the associated data. The personal information optionally comprises social networking information and optionally comprises family tree information. In some cases, the fitness data comprises data generated by a fitness tracking device or input by the user into a fitness log. In such cases, the fitness data may be ingested by accessing an API or by upload of one or more data files provided by the individual. In some cases, the genetic data comprises nucleic acid sequence information. In such cases, the nucleic acid sequence information comprises DNA sequence information or RNA sequence information. The genetic data may be ingested by accessing an API or by upload of one or more data files provided by the individual. In some cases, the medical data comprises at least one electronic health record (EHR) or at least one personal health record (PHR). In such cases, the medical data is ingested by accessing an API or by upload of one or more data files provided by the individual. The application may further comprise a software module providing a coupon, discount, or subsidy for genetic sequencing services in response to the ingesting of the fitness data, the medical data, or both the fitness data and the medical data. Also, the application may further comprise a software module generating and presenting a genetic data summary for the individual, the genetic data summary comprising a plurality of phenotypes and further comprising at least one annotation, at least one article, and a confidence level associated with each phenotype. In some cases, the application further comprises a software module applying an algorithm to generate a monetary market value for the profile and the associated data. In such cases, the monetary market value is based, at least in part, on one or more of: the quantity of the data, the quality of the data, the number of types of data, the number of family members of the individual who have profiles, and whether a health trust is established. The application is optionally implemented as software-as-a-service (SaaS).

In another aspect, disclosed herein are computer-implemented methods for providing a marketplace for health information: maintaining a database of profiles, each profile for an individual and comprising personal information, fitness data, genetic data, and medical data for the individual; applying an algorithm to generate a monetary market value for each profile; providing tools for a health data consumer to conduct a search of the database by inputting one or more phenotypes, the search resulting in one or more identified profiles; and providing tools for the health data consumer to select one or more of the identified profiles and subscribe to the selected profiles. In various cases, the database of profiles comprises at least 1,000 profiles, at least 10,000 profiles, or at least 100,000 profiles. The tools for a health data consumer to conduct a search of the database may optionally allow the health data consumer to refine the search by inputting one or more single-nucleotide polymorphisms (SNPs) associated with the one or more phenotypes. The algorithm may, in some cases, generate the monetary market value for each profile based, at least in part, on one or more of: the personal information, the quantity of the fitness data, the quality of the fitness data, the quantity of the genetic data, the quality of the genetic data, the quantity of the medical data, the quality of the medical data, the number of types of data in the profile, and the number of family members of the individual who have profiles in the database. The method may further comprise notifying each individual when a health data consumer subscribes to their profile. In some cases, subscribing to a profile requires payment by the health data consumer based on the monetary market value for the profile. In such cases, the method may further comprise compensating each individual, based on the monetary market value for their profile, when a health data consumer subscribes to their profile. The subscriptions are optionally documented and managed with blockchain technology. Also, in some cases, the method further comprises providing tools for the heath data consumer to contact an individual to whose profile the heath data consumer is subscribed. The method may further comprise generating a recommendation comprising one or more profiles for the data consumer.

In another aspect, disclosed herein are computer-implemented systems comprising: a digital processing device comprising: at least one processor, an operating system configured to perform executable instructions, a memory, and a computer program including instructions executable by the digital processing device to create a health information marketplace application comprising: a database of profiles, each profile for an individual and comprising personal information, fitness data, genetic data, and medical data for the individual; a software module applying an algorithm to generate a monetary market value for each profile; a software module presenting an interface for allowing a health data consumer to conduct a search of the database by inputting one or more phenotypes, the search resulting in one or more identified profiles; and a software module presenting an interface for allowing the health data consumer to select one or more of the identified profiles and subscribe to the selected profiles. In various cases, the database of profiles comprises at least 1,000 profiles, at least 10,000 profiles, or at least 100,000 profiles. The interface for allowing a health data consumer to conduct a search of the database of profiles may optionally allow the health data consumer to refine the search by inputting one or more single-nucleotide polymorphisms (SNPs) associated with the one or more phenotypes. The software module applying an algorithm to generate a monetary market value for each profile may, in some cases, generate the monetary market value based, at least in part, on one or more of: the personal information, the quantity of the fitness data, the quality of the fitness data, the quantity of the genetic data, the quality of the genetic data, the quantity of the medical data, the quality of the medical data, the number of types of data in the profile, and the number of family members of the individual who have profiles in the database. The application may further comprise a software module notifying each individual when a health data consumer subscribes to their profile. In some cases, subscribing to a profile requires payment by the health data consumer based on the monetary market value for the profile. In such cases, the application further comprises a software module compensating each individual, based on the monetary market value for their profile, when a health data consumer subscribes to their profile. The subscriptions are optionally documented and managed with blockchain technology. Also, the application may further comprise a software module allowing the heath data consumer to contact an individual to whose profile the heath data consumer is subscribed. In some cases, the application further comprises a software module generating a recommendation comprising one or more profiles for the data consumer. The application is optionally implemented as software-as-a-service (SaaS).

In another aspect, disclosed herein are non-transitory computer-readable storage media encoded with a computer program including instructions executable by one or more processors to create a health information marketplace application comprising: a database of profiles, each profile for an individual and comprising personal information, fitness data, genetic data, and medical data for the individual; a software module applying an algorithm to generate a monetary market value for each profile; a software module presenting an interface for allowing a health data consumer to conduct a search of the database by inputting one or more phenotypes, the search resulting in one or more identified profiles; and a software module presenting an interface for allowing the health data consumer to select one or more of the identified profiles and subscribe to the selected profiles. In various cases, the database of profiles comprises at least 1,000 profiles, at least 10,000 profiles, or at least 100,000 profiles. The interface for allowing a health data consumer to conduct a search of the database of profiles may optionally allow the health data consumer to refine the search by inputting one or more single-nucleotide polymorphisms (SNPs) associated with the one or more phenotypes. The software module applying an algorithm to generate a monetary market value for each profile may, in some cases, generate the monetary market value based, at least in part, on one or more of: the personal information, the quantity of the fitness data, the quality of the fitness data, the quantity of the genetic data, the quality of the genetic data, the quantity of the medical data, the quality of the medical data, the number of types of data in the profile, and the number of family members of the individual who have profiles in the database. The application may further comprise a software module notifying each individual when a health data consumer subscribes to their profile. In some cases, subscribing to a profile requires payment by the health data consumer based on the monetary market value for the profile. In such cases, the application further comprises a software module compensating each individual, based on the monetary market value for their profile, when a health data consumer subscribes to their profile. The subscriptions are optionally documented and managed with blockchain technology. Also, the application may further comprise a software module allowing the heath data consumer to contact an individual to whose profile the heath data consumer is subscribed. In some cases, the application further comprises a software module generating a recommendation comprising one or more profiles for the data consumer. The application is optionally implemented as software-as-a-service (SaaS).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a non-limiting example of a graphic user interface; in this case, an interface for inputting personal information to associate with an individual profile;

FIG. 2 shows a non-limiting example of a graphic user interface; in this case, an interface for configuring ingestion of fitness data to associate with an individual profile;

FIG. 3 shows a non-limiting example of a graphic user interface; in this case, an interface for configuring ingestion of fitness data from a specific fitness tracking service provider;

FIG. 4 shows a non-limiting example of a ingested fitness data; in this case, cycling data;

FIGS. 5 and 6 show non-limiting examples of graphic user interfaces; in this case, interfaces for configuring ingestion of genetic data to associate with an individual profile;

FIG. 7 shows a non-limiting example of a graphic user interface; in this case, an interface for configuring ingestion of genetic data from specific genetic sequencing service providers;

FIG. 8 shows a non-limiting example of a ingested genetic data; in this case, DNA sequence data presented as a genetic summary reflecting specific phenotypes associated with specific SNPs of the individual;

FIG. 9 shows a non-limiting example of a graphic user interface; in this case, an interface for configuring ingestion of medial data, in the form of EHRs or PHRs to associate with an individual profile;

FIG. 10 shows a non-limiting example of a graphic user interface; in this case, an interface for configuring ingestion of EHRs from specific medical service providers;

FIG. 11 shows a non-limiting example of ingested medical data; in this case, EHR data;

FIG. 12 shows a non-limiting example of a graphic user interface; in this case, an interface for inputting personal health information to supplement PHRs associated with a profile;

FIG. 13 shows a non-limiting example of a graphic user interface; in this case, an interface providing a personal health data dashboard with an integrated view;

FIG. 14 shows a non-limiting example of a graphic user interface; in this case, an interface providing a personal health data dashboard with a fitness data view;

FIG. 15 shows a non-limiting example of a graphic user interface; in this case, an interface providing a personal health data dashboard with a first genetic data view;

FIG. 16 shows a non-limiting example of a graphic user interface; in this case, an interface providing a personal health data dashboard with a second genetic data view;

FIG. 17 shows a non-limiting example of a graphic user interface; in this case, an interface providing a personal health data dashboard with a medical data view;

FIG. 18 shows a non-limiting example of a graphic user interface; in this case, an interface providing a personal health data dashboard with a timeline view;

FIG. 19 shows a non-limiting example of a health trust certificate; in this case, health trust certificate indicating that personal, fitness, DNA, and health data are associated with the profile that has been placed in trust;

FIG. 20 shows a non-limiting example of a graphic user interface; in this case, an interface for designating beneficiaries for a health trust;

FIG. 21 shows a non-limiting example of a graphic user interface; in this case, an interface for managing a health trust including features for monitoring recipients, managing recipients, adding recipients, and viewing a health trust certificate;

FIG. 22 shows a non-limiting example of a suitable high-level system architecture;

FIG. 23 shows a non-limiting example of a suitable application layer architecture;

FIG. 24 shows a non-limiting example of a digital processing device; in this case, a device with one or more CPUs, a memory, a communication interface, and a display;

FIG. 25 shows a non-limiting example of a web/mobile application provision system; in this case, a system providing browser-based and/or native mobile user interfaces; and

FIG. 26 shows a non-limiting example of a cloud-based web/mobile application provision system; in this case, a system comprising an elastically load balanced, auto-scaling web server and application server resources as well synchronously replicated databases.

### DETAILED DESCRIPTION OF THE INVENTION

Described herein are computer-implemented methods of managing health information: providing tools for an individual to create a profile, the profile comprising personal information; ingesting fitness data, genetic data, and medical data (e.g., electronic and/or paper health records) for the individual and associating the fitness data, the genetic data, and the medical data, including electronic and/or paper health records, with the profile; and providing tools for the individual to designate one or more recipient successors for all or part of the profile and the associated data to create a health trust.

Also described herein are non-transitory computer-readable storage media encoded with a computer program including instructions executable by a processor to create a health dashboard application comprising: a software module presenting an interface for creating a profile for an individual, the profile comprising personal information; a software module ingesting fitness data, genetic data, and medical data (e.g., electronic and/or paper health records) for the individual and associating the fitness data, the genetic data, and the medical data, including electronic and/or paper health records, with the profile; and a software module presenting an interface for allowing the individual to designate one or more recipient successors for all or part of the profile and the associated data to create a health trust.

Also described herein are computer-implemented systems comprising: a digital processing device comprising: at least one processor, an operating system configured to perform executable instructions, a memory, and a computer program including instructions executable by the digital processing device to create a health dashboard application comprising: a software module presenting an interface for creating a profile for an individual, the profile comprising personal information; a software module ingesting fitness data, genetic data, and medical data (e.g., electronic and/or paper health records) for the individual and associating the fitness data, the genetic data, and the medical data, including electronic and/or paper health records, with the profile; and a software module presenting an interface for allowing the individual to designate one or more recipient successors for all or part of the profile and the associated data to create a health trust.

Also described herein are computer-implemented methods for providing a marketplace for health information: maintaining a database of profiles, each profile for an individual and comprising personal information, fitness data, genetic data, and medical data, such as electronic and/or paper medical records, for the individual; applying an algorithm to generate a monetary market value for each profile; providing tools for a health data consumer to conduct a search of the database by inputting one or more phenotypes, the search resulting in one or more identified profiles; and providing tools for the health data consumer to select one or more of the identified profiles and subscribe to the selected profiles.

Also described herein are computer-implemented systems comprising: a digital processing device comprising: at least one processor, an operating system configured to perform executable instructions, a memory, and a computer program including instructions executable by the digital processing device to create a health information marketplace application comprising: a database of profiles, each profile for an individual and comprising personal information, fitness data, genetic data, and medical data for the individual; a software module applying an algorithm to generate a monetary market value for each profile; a software module presenting an interface for allowing a health data consumer to conduct a search of the database by inputting one or more phenotypes, the search resulting in one or more identified profiles; and a software module presenting an interface for allowing the health data consumer to select one or more of the identified profiles and subscribe to the selected profiles.

Also described herein are non-transitory computer-readable storage media encoded with a computer program including instructions executable by one or more processors to create a health information marketplace application comprising: a database of profiles, each profile for an individual and comprising personal information, fitness data, genetic data, and medical data (e.g., electronic and/or paper medical records) for the individual; a software module applying an algorithm to generate a monetary market value for each profile; a software module presenting an interface for allowing a health data consumer to conduct a search of the database by inputting one or more phenotypes, the search resulting in one or more identified profiles; and a software module presenting an interface for allowing the health data consumer to select one or more of the identified profiles and subscribe to the selected profiles.

### Certain Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, “EFGHR” refers to an electronic aggregated health record, which includes fitness, genetic, and health information for an individual or a population, group, or cohort of individuals. “EFGHR” is optionally used as an acronym for “electronic fitness, genetic, and health record.”

As used herein, “health trust” refers to a health information protection and succession planning instrument allowing an individual to designate one or more successors for the health information placed in trust. A health trust may encompass fitness, genetic, and medical information including genetic data (e.g., DNA and/or RNA sequence data, variant data, and epigenetic data), metabolomic data, proteomic data, microbiomic data, medical history, medication record, medication history, authenticated physical exams, laboratory test reports, imaging reports, family history, allergies, adverse drug reactions, illnesses, chronic diseases, hospitalizations, surgeries, immunization status, vital signs, and other biometrics. A health trust may encompass health information for an individual or a group of related individuals (e.g., a family).

### Individual Profiles

The platforms, systems, media, and methods described herein include one or more databases, or other data stores, of profiles, or use of the same. Each of the profiles is associated with an individual. In some cases, profiles may be associated with each other to represent or indicate a relationship between individuals, such as a familial relationship. By way of example, profiles may be associated hierarchically to represent the relationships of a family tree. By way of further example, profiles may be associated to represent a health trust, described further herein, that includes a trustee and one or more designated recipient successors for the trustee profile and the data associated therewith.

Data pertaining to an individual is associated with each profile. Many types of data are suitable for association with a profile. By way of examples, personal information, fitness data, genetic data, and medical data, such as electronic and/or paper medical records, all described herein, for the individual are suitably associated with the individual's profile. By way of further examples, health directives, wills, powers of attorney, and estate planning trusts for the individual are suitably associated with the individual's profile.

The applications described herein suitably include, for example, more than 100 profiles, more than 1,000 profiles, more than 10,000, profiles, more than 50,000 profiles, more than 100,000 profiles, more than 500,000 profiles, more than 1,000,000 profiles, more than 10,000,000 profiles, or more than 100,000,000 profiles, including increments therein. In light of the disclosure provided herein, those of skill in the field will recognize that the personal data marketplace described herein will benefit from larger numbers of profiles. Greater depth and diversity of profiles, and information associated therewith, creates a more lively and useful marketplace.

Preferably, the profiles are searchable and/or indexed to facilitate finding particular individuals and particular data. In some cases, the profiles, and the data associated therewith, are encrypted and/or anonymized to increase privacy and security.

### Personal Information

The platforms, systems, media, and methods described herein include personal information, or use of the same. Personal information for an individual may be associated with a profile for that individual. Many types of personal information are suitable for association with a profile. Suitable personal information includes, by way of non-limiting examples, name, age, gender, date of birth, address, height, weight, race, ethnicity, demographic information, marital status, family status, family tree, sexual orientation, social networking profiles, accounts, and connections, and the like.

Personal information may be ingested by the applications described herein in a variety of ways. In some cases, a user enters personal information via a web or mobile application that includes an interface for personal information entry. In other cases, the user may identify an online source of personal information, such as a professional networking or social media profile, which can be accessed to obtain personal information. In still other cases, the applications described herein access an API offered by a third-party, with permission of the individual, to ingest personal information. In further cases, the applications described herein are pre-linked with third-party data stores and service providers to facilitate ingestion of personal information if and when an individual grants permission to access the information.

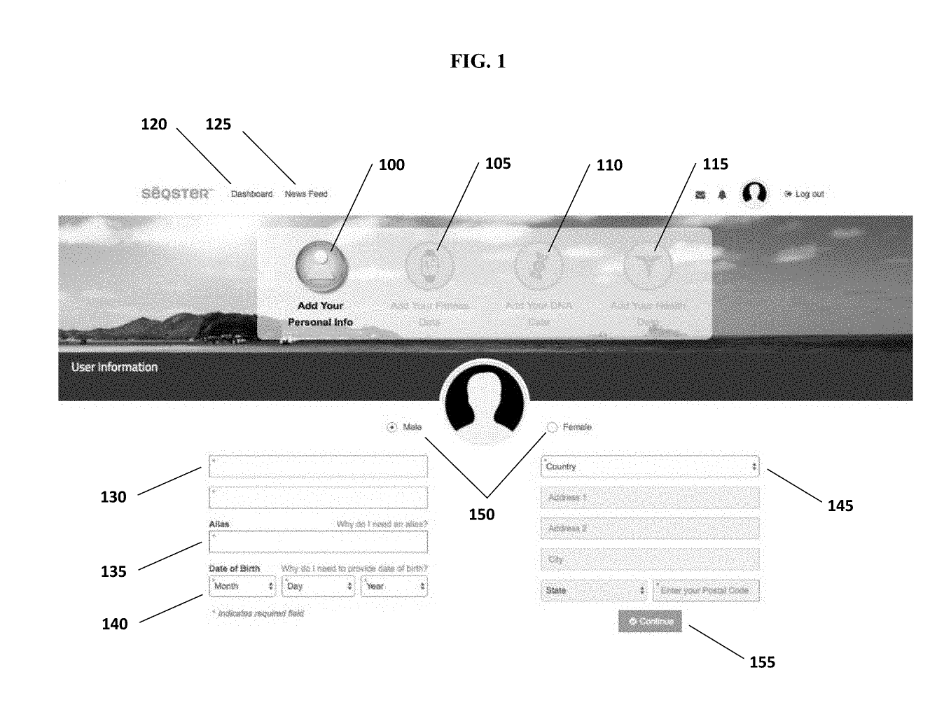

Referring to FIG. 1, an exemplary interface for inputting personal information to associate with an individual profile includes an information entry progress indicator, which shows what types of information the individual has provided. In this example, the progress indicator shows whether the individual has provided personal information 100, fitness data 105, DNA data 110, and heath data 115. Continuing to refer to FIG. 1, the interface includes links to a dashboard for the individual 120 and a news feed for the individual 125. The interface for inputting personal information further includes text fields for entry of first name and last name 130, alias 135, date of birth 140, location (e.g., country, street address, city, state, and postal code) 145, and gender 150. When the individual is done inputting their personal information, they interact with a “Continue” button 155 to associate the entered information with their profile.

### Fitness Data

The platforms, systems, media, and methods described herein include fitness data, or use of the same. Fitness data for an individual may be associated with a profile for that individual. Many types of fitness data are suitable for association with a profile. Suitable fitness data includes, by way of non-limiting examples, exercise data, physiological data, and the like. Exercise data may include raw data, data organized by exercise events, data organized by exercise type, or exercise trends for the individual. Where exercise data includes exercise events, the data can indicate frequency of events, duration of events, speed and/or distance (if applicable), intensity of events, as well as the type of exercise. Physiological data pertaining to fitness may include average heart rate, maximum heart rate blood pressure, respiration rate, VO₂ max, and the like, for any period of time. The fitness data may be generated by a fitness measurement/tracking device, such as a wearable device. Many wearable fitness devices are suitable, including, by way of non-limiting examples, those offered by Fitbit®, Garmin®, Misfit®, Apple®, Samsung®, Huawei®, Withings®, Polar®, Timex®, Athos®, TomTom®, Sony®, Pebble®, and the like.

Fitness data may be ingested by the applications described herein in a variety of ways. In some cases, a user enters fitness data via a web or mobile application that includes an interface for fitness data entry. In other cases, the user may identify an online source of fitness data, such as a fitness log or blog, a fitness tracking application, or a fitness tracking device data store, which can be accessed to obtain fitness data. In still other cases, the applications described herein access an API offered by a third-party, with permission of the individual, to ingest fitness data. In further cases, the applications described herein are pre-linked with third-party data stores and service providers to facilitate ingestion of fitness data if and when an individual grants permission to access the data.

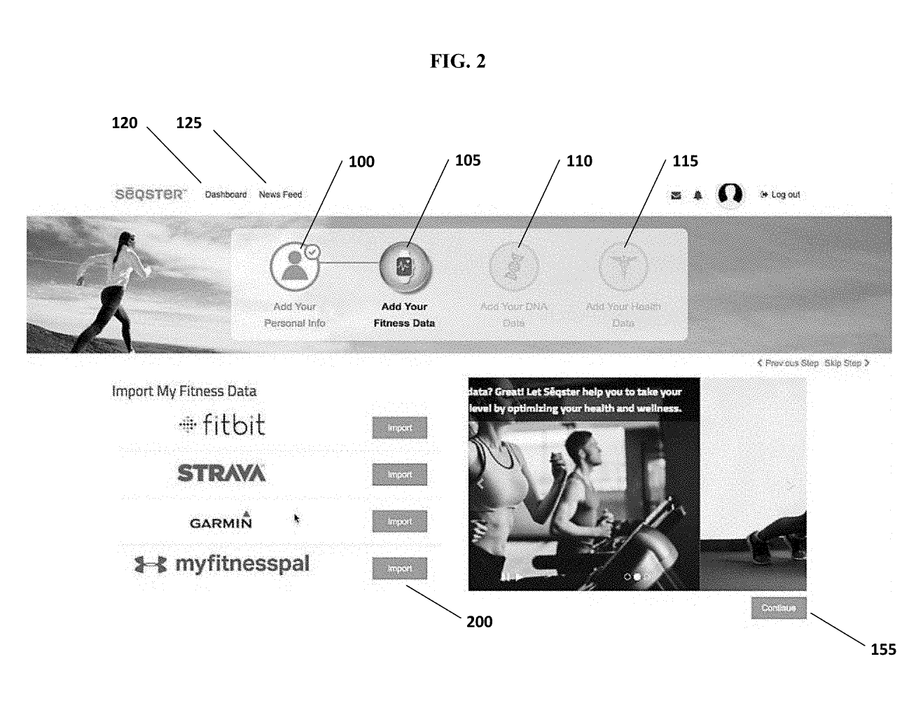

Referring to FIG. 2, an exemplary interface for ingesting fitness data to associate with an individual profile includes an information entry progress indicator, which shows what types of information the individual has provided. In this example, the progress indicator shows that the individual has provided personal information 100, is in the process of providing fitness data 105, but has not yet provided DNA data 110 or heath data 115. The interface includes elements to access, and import fitness data from, multiple third-party fitness tracking device/service providers storing fitness data and offering APIs to access the data online. The individual may begin a fitness data import process by, for example, interacting with an “Import” button 200 associated with one of the identified sources of stored fitness data. Once the source of the fitness data is identified, the individual may interact with a “Continue” button 155 to advance the ingestion process.

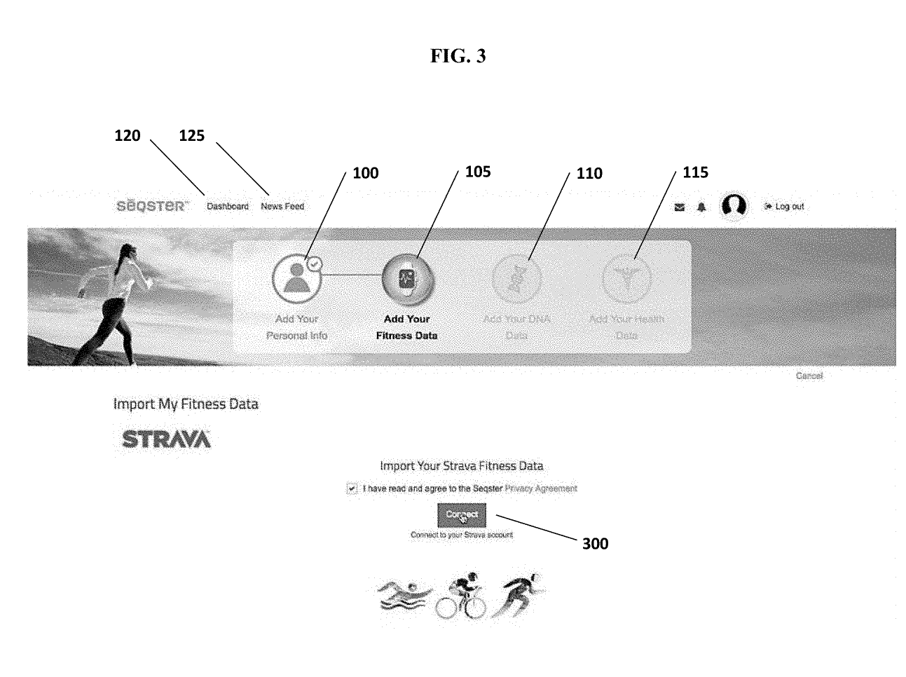

Referring to FIG. 3, an exemplary interface for ingesting fitness data to associate with an individual profile includes a link to review privacy information to explain to the individual how the fitness data will be used. After acknowledging the privacy agreement, the user may interact with a “Connect” button 300 to further advance the ingestion process.

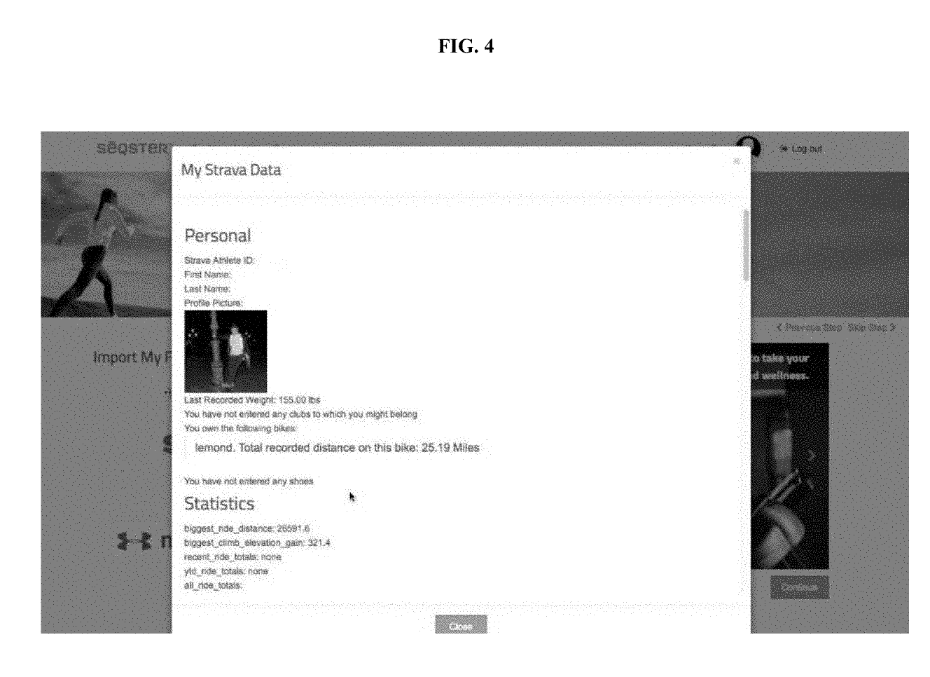

Referring to FIG. 4, the individual may review and/or update ingested fitness data at any time. The individual optionally uses the interface to access and view ingested fitness data. In this example, the fitness data is cycling data and includes first name, last name, a profile picture, last recorded weight, fitness clubs to which the individual belongs, fitness equipment owned by the individual, and fitness statistics. The cycling statistics include longest ride distance, largest elevation gain, recent ride totals, year to date ride totals, and historic ride totals.

### Genetic Data

The platforms, systems, media, and methods described herein include genetic data, or use of the same. Genetic data for an individual may be associated with a profile for that individual. Many types of genetic data are suitable for association with a profile. Suitable genetic data includes, by way of non-limiting examples, nucleic acid data, such as DNA data and/or RNA data, epigenetic data, or the like. In some cases, the nucleic acid data includes sequence data, methylation data, expression data, or the like. DNA sequence data suitably include whole genome sequence data, partial genome sequence data (e.g., sequence data for one or more genes), whole exome sequence data, partial exome sequence data, or the like.

The genetic data may include nucleic acid sequence mutation or variant data derived from comparing a nucleic acid sequence of the individual to one or more references such as a reference genome. The mutation or variant data may include data on substitutions (including point mutations and single-nucleotide polymorphisms (SNPs)), insertions, deletions, missense mutations, nonsense mutations, repeat expansions, and/or frameshifts in one or more genes of the individual. In some cases, the genetic data includes a genotype based on SNPs present in one or more genes of the individual.

Genetic data may be ingested by the applications described herein in a variety of ways. In some cases, a user uploads genetic data via a web or mobile application that includes an interface for genetic data file upload. In other cases, the user may identify an online source of genetic data, such as a health or genealogy service provider or a genetic data store, which can be accessed to obtain genetic data. In still other cases, the applications described herein access an API offered by a third-party, with permission of the individual, to ingest genetic data. In further cases, the applications described herein are pre-linked with third-party data stores and service providers to facilitate ingestion of genetic data if and when an individual grants permission to access the data.

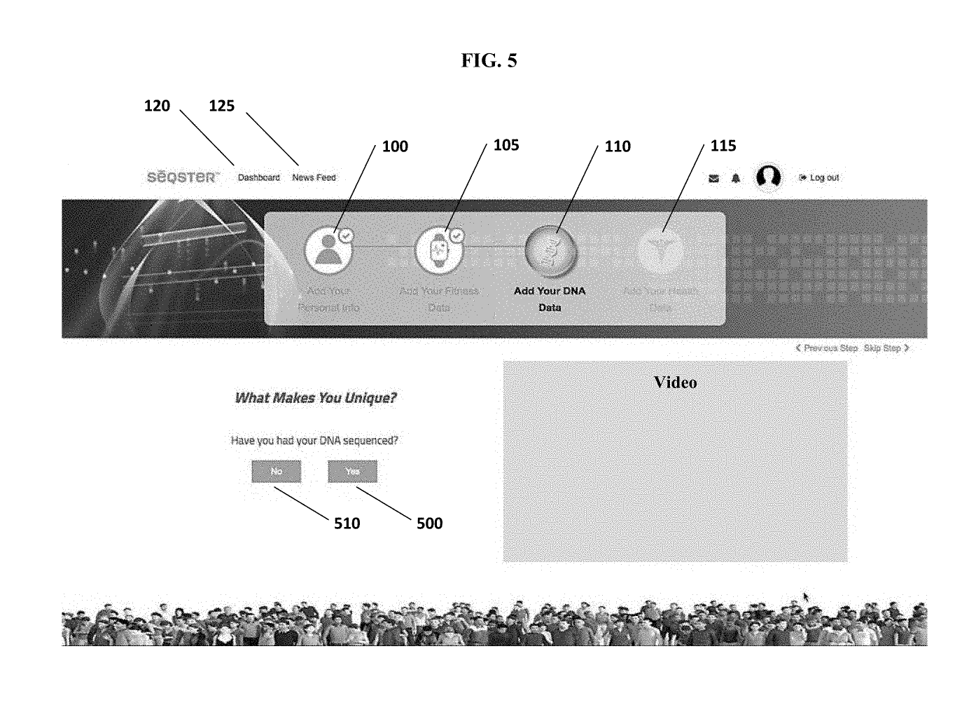

Referring to FIG. 5, an exemplary interface for ingesting genetic data to associate with an individual profile includes an information entry progress indicator, which shows what types of information the individual has provided. In this example, the progress indicator shows that the individual has provided personal information 100 and fitness data 105, is in the process of providing DNA data 110, but has not yet provided heath data 115. The interface for ingesting genetic data to associate with an individual profile, in some cases, includes a preliminary survey question asking an individual whether they have had their DNA sequenced. The individual may advance the ingestion process by interacting with either a “No” button 510 or a “Yes” button 500 to answer the preliminary survey question.

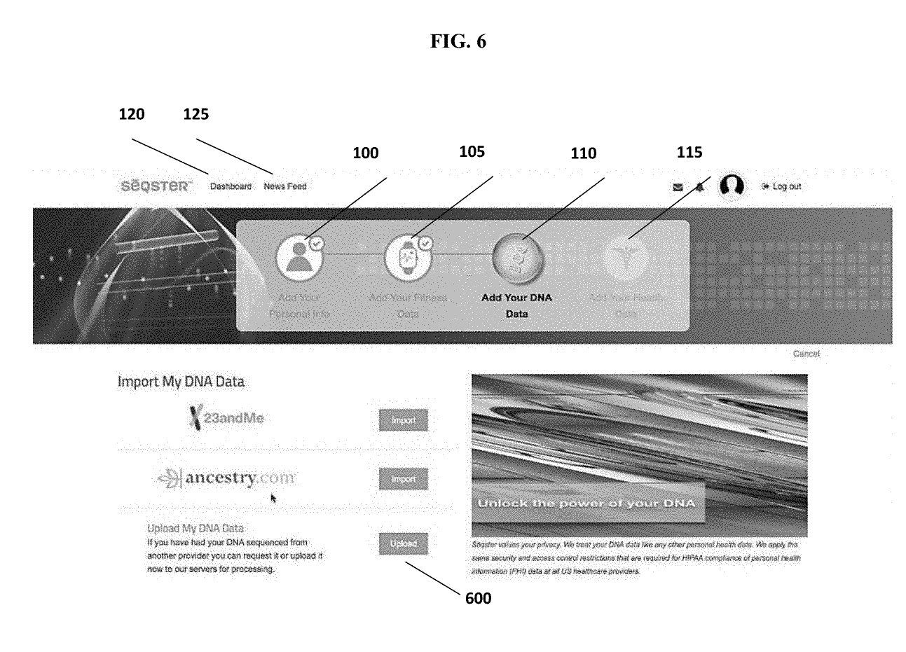

Referring to FIG. 6, an exemplary interface for ingesting genetic data to associate with an individual profile includes elements to access, and import genetic data from, multiple third-party health and genealogy service providers storing genetic data and offering APIs to access the data online. The individual may begin a genetic data import process by, for example, interacting with an “Import” button 600 associated with one of the identified sources of stored genetic data.

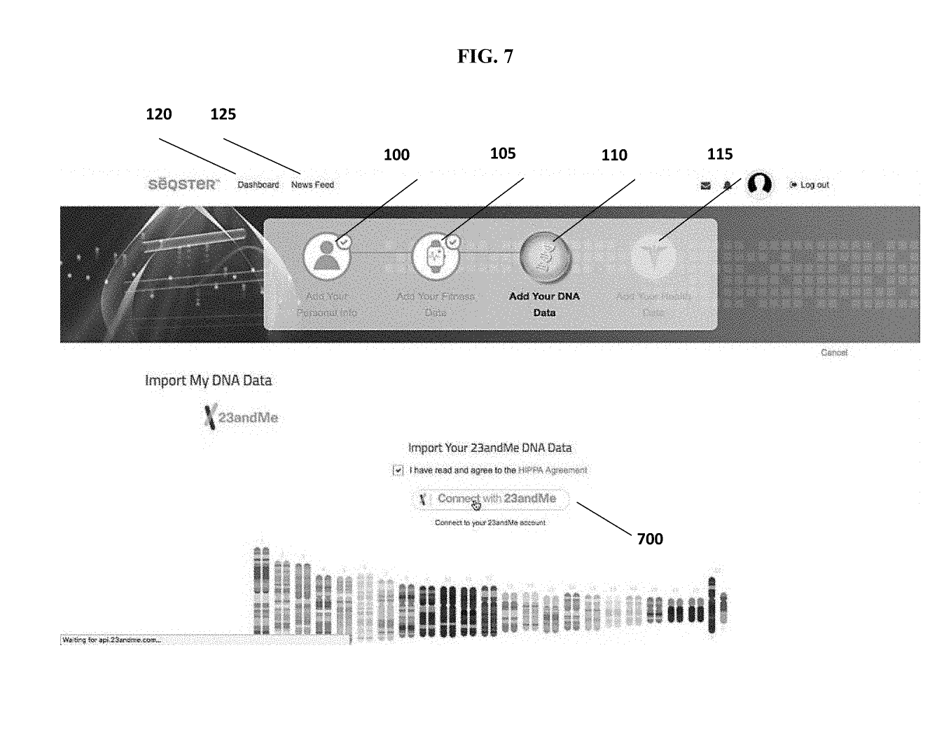

Referring to FIG. 7, an exemplary interface for ingesting genetic data to associate with an individual profile includes a link to review HIPAA information to explain to the individual how the genetic data will be used and to gain the individual's permission to access the genetic data. After acknowledging the HIPAA agreement, the user may interact with a “Connect” button 700 to further advance the ingestion process.

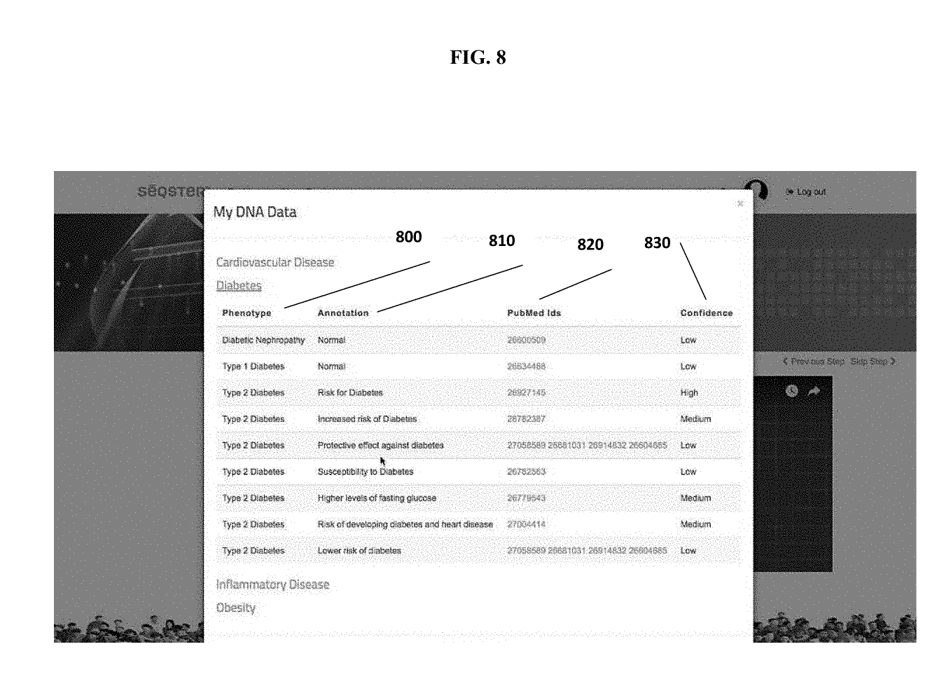

Referring to FIG. 8, the individual may review and/or update ingested genetic data at any time. The individual optionally uses the interface to access and view ingested genetic data. In this example, the genetic data is presented in the form of a genetic data summary. The genetic data summary may include information based on the SNPs or other variations identified for the individual in the genetic data. For example, the genetic data summary may include a plurality of phenotypes 800 associated with SNPs or other variations identified for the individual. In this example, for each phenotype annotations 810, articles 820 (in the form of, for example, PubMed IDs, etc.), and a confidence level 830 for the association (in the form of, for example, a percentage, a rating, a ranking, etc.) are provided in the summary.

### Medical Data

The platforms, systems, media, and methods described herein include medical data, or use of the same. Medical data for an individual may be associated with a profile for that individual. Many types of medical data are suitable for association with a profile. Suitable medical data includes, by way of non-limiting examples, electronic health records (EHRs), personal health records (PHRs), and the like.

In view of the disclosure provided herein, those of skill in the art will recognize that an EHR is a systematic collection of electronic health information about an individual or population. In some embodiments, an EHR includes records of therapies, prescriptions, orders, or instructions issued by a healthcare provider for an individual. EHRs suitable for use with the systems, devices, software, and methods disclosed herein optionally include a range of data in comprehensive or summary form, including, by way of non-limiting examples, metabolomic data, proteomic data, microbiomic data, medical history, medication record, medication history, authenticated physical exam, laboratory test reports (e.g., pathology report, blood cell count report, blood culture report, urinalysis report, throat culture report, genetic test report, etc.), imaging reports (e.g., X-ray, CT scan, MRI, ultrasound, etc.), demographics, family history, allergies, adverse drug reactions, illnesses, chronic diseases, hospitalizations, surgeries, immunization status, vital signs and other biometrics (e.g., body temperature, heart rate, blood pressure, respiratory rate, blood diagnostics such as oxygen saturation, glucose concentration, and blood count, urine diagnostics such as specific gravity, protein, glucose, and blood, other bodily fluid diagnostics, diagnostic images or imaging reports, etc.), age, weight, Observations of Daily Living (ODLs), insurance benefits, insurance, eligibility, insurance claim information, and billing information.

In view of the disclosure provided herein, those of skill in the art will recognize that suitable EHRs include those created and maintained in accordance with published standards, including XML-based standards such as Continuity of Care Record (CCR). Suitable EHRs also include those utilizing the DICOM communications protocol standard for representing and transmitting radiology (and other) image-based data, the HL7 standardized messaging and text communications protocol, and ANSI X12 transaction protocols for transmitting individual and billing data. Additionally, those in the art will recognize that suitable EHRs include those operable with open standard specifications that describe the management, storage, retrieval, and exchange of health data, such as openEHR (available at http://www.openehr.org/).

In view of the disclosure provided herein, those of skill in the art will recognize that a personal health record (PHR) is a health record where health data and information related to the care of an individual is maintained by the individual. This stands in contrast to EHRs, which are typically maintained by institutions. PHRs suitably include information pertaining to allergies and adverse drug reactions, chronic diseases, family history, illnesses and hospitalizations, imaging reports, laboratory test results, medications and dosing, prescription record, surgeries and other procedures, vaccinations, and observations of daily living (ODLs), etc. PHRs also may include records of electronic messaging between patients and providers and records of medical appointments. In some cases, suitable PHRs are created by an individual entering information directly, either by typing into web- or mobile-based forms or uploading/transmitting data from a file or another web-based storage.

Medical data may be ingested by the applications described herein in a variety of ways. In some cases, a user uploads medical data via a web or mobile application that includes an interface for medical data file upload. In other cases, the user may identify an online source of medical data, such as a health care provider or a medical data store, which can be accessed to obtain medical data. In still other cases, the applications described herein access an API offered by a third-party, with permission of the individual, to ingest medical data. In further cases, the applications described herein are pre-linked with third-party data stores and service providers to facilitate ingestion of medical data if and when an individual grants permission to access the data.

An individual authorizes access to their health records. To facilitate authorization, the applications described herein include a software module for verifying an individual's authorization to access their health records. In some embodiments, the authorization meets applicable legal requirements. In further embodiments, the applicable legal requirements include, by way of non-limiting examples, those in the Health Insurance Portability and Accountability Act of 1996 and the Health Information Technology for Economic and Clinical Health Act of 2009.

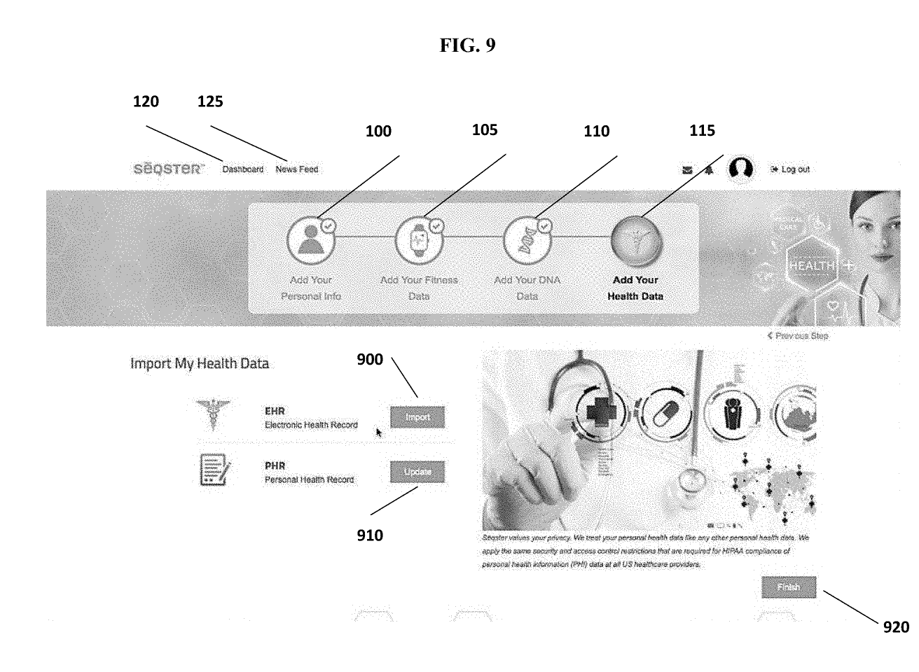

Referring to FIG. 9, an exemplary interface for ingesting medical data to associate with an individual profile includes an information entry progress indicator, which shows what types of information the individual has provided. In this example, the progress indicator shows that the individual has provided personal information 100, fitness data 105, and DNA data 110, and is in the process of providing health data 115. The interface for ingesting medical data to associate with an individual profile, in some cases, includes an interface element allowing an individual to import EHRs by, for example, interacting with an “Import” button 900. Similarly, the interface for ingesting medical data to associate with an individual profile, in some cases, includes an interface element allowing an individual to import PHRs by, for example, interacting with an “Import” button 910. Once the sources of the medical data are identified, the individual may interact with a “Continue” button 920 to advance the ingestion process.

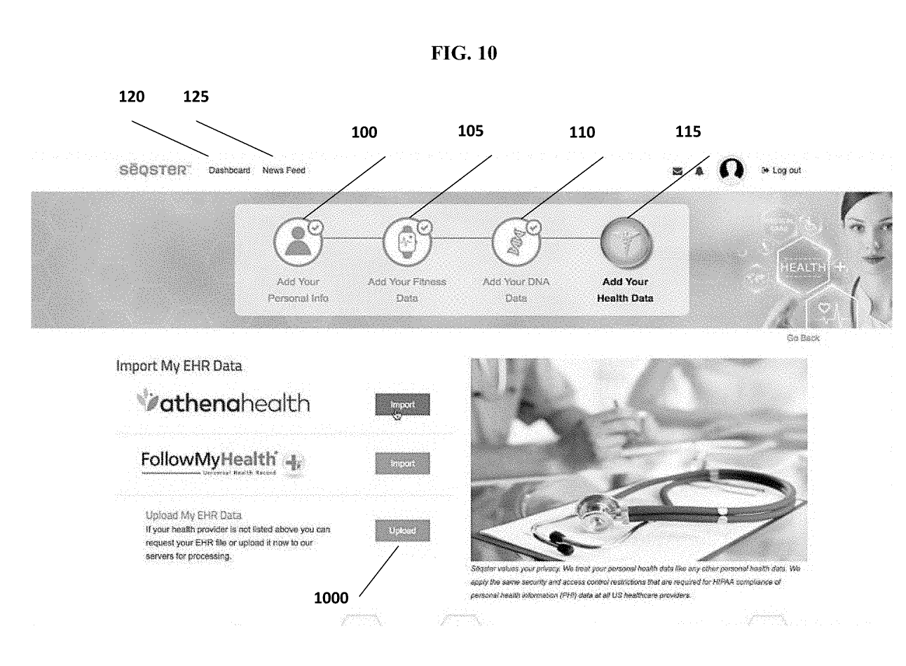

Referring to FIG. 10, an exemplary interface for ingesting medical data includes elements to access and import EHRs from multiple third-party healthcare-related service providers storing EHRs and offering APIs to access the data online. By way of example, an individual may begin an EHR import process by, for example, interacting with an “Import” button associated with one of the identified sources of EHRs. By way of further example, an individual may begin an EHR import process by, for example, interacting with an “Upload” button 1000 to request EHRs from a healthcare-related service provider.

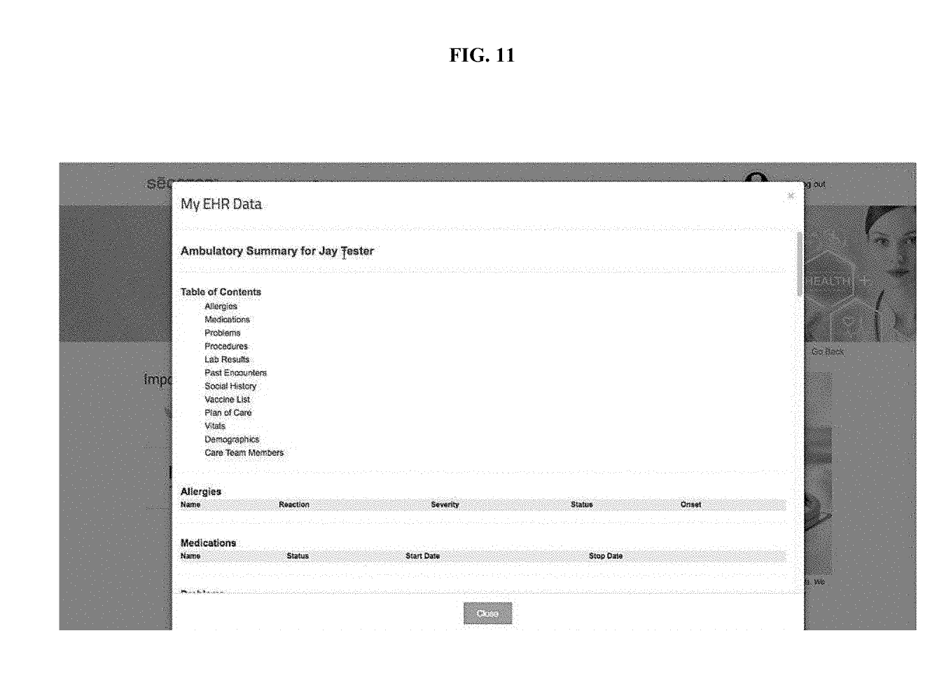

Referring to FIG. 11, the individual may review and/or update EHRs at any time. The individual optionally uses the interface to access and view ingested EHRs. In this example, EHRs are presented in the form of an EHR summary. For example, an EHR summary may include allergies, medications, problems, procedures, lab results, past encounters, social history, vaccines, plan of care, vital signs, demographics, and care team members for an individual.

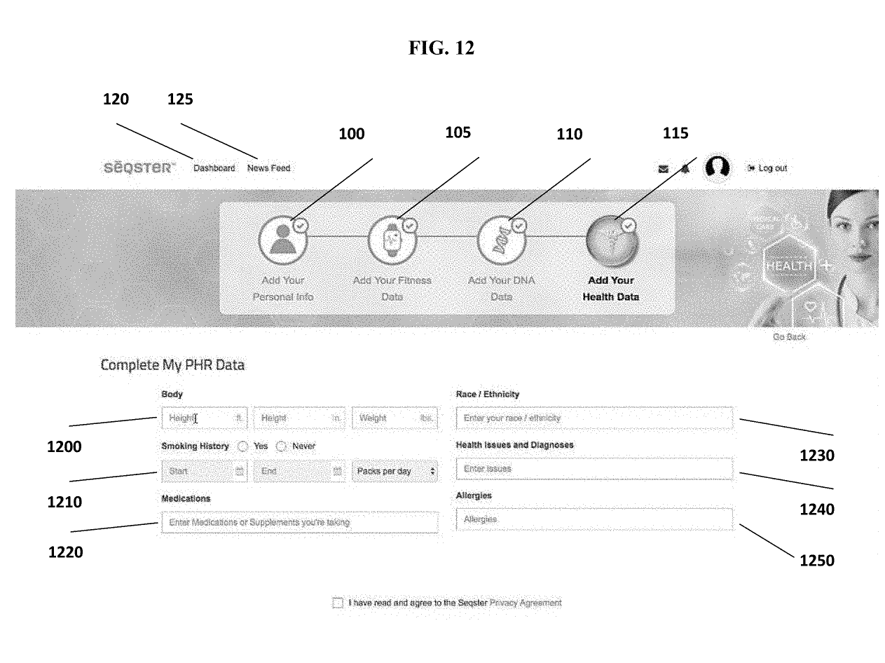

Referring to FIG. 12, an exemplary interface for inputting PHR data to associate with an individual profile includes interface elements for entry of height and weight 1200, smoking history, 1210, medications 1220, race/ethnicity 1230, health issues and diagnoses 1240, and allergies 1250. When the individual is done inputting their PHR data, they interact with an interface element to acknowledge a privacy agreement to advance the input process.

### Health Dashboard

The platforms, systems, media, and methods described herein include a health dashboard, or use of the same. In some cases the health dashboard includes tools, health data summaries, and health data visualizations and a set of navigation elements to allow users to access these features.

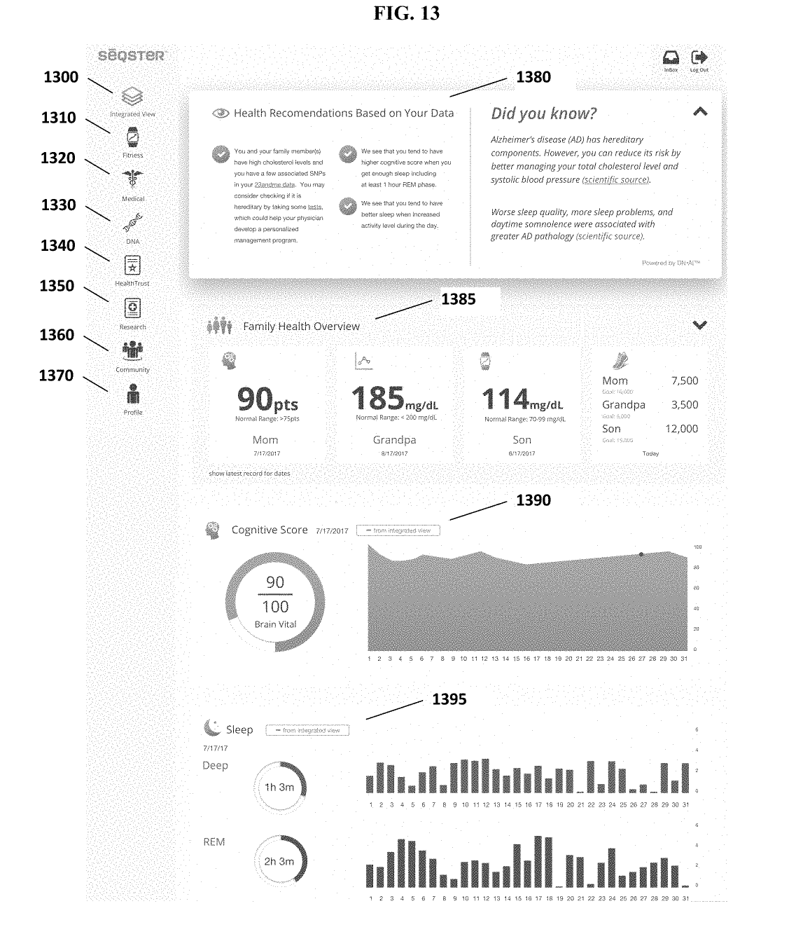

Referring to FIG. 13, the health dashboard may include an integrated view. In this example, the health dashboard includes a navigation bar providing optional access to: the integrated view 1300, a fitness view 1310, a medical view 1320, a DNA view 1330, a health trust view 1340, a medical research view 1350, a community view 1360, and a user profile view 1370. The health dashboard, in this case, includes health recommendations based on the user's health data 1380. These recommendations can be based on the users, fitness data, genetic data, or medical data, or any combinations thereof. Further, in this example, the health dashboard includes a family health overview 1385, which lists significant new events for all profiles linked by familial relations as well as summaries of recent cognitive data 1390 and sleep data 1395.

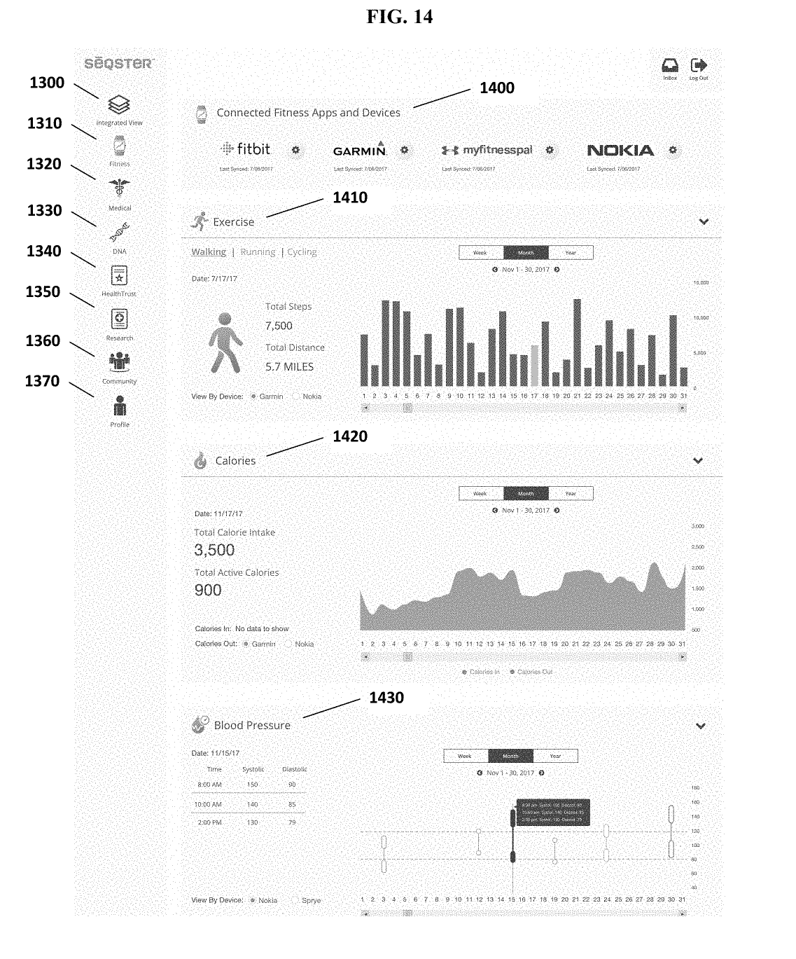

Referring to FIG. 14, the health dashboard may include one or more fitness data views. In this example, the health dashboard includes a list of connected fitness apps and devices, including wearable devices 1400. Further, in this example, the health dashboard includes summaries of specific relevant fitness-related health data such as an exercise data summary 1410, a calorie burn summary 1420, and a blood pressure data summary 1430.

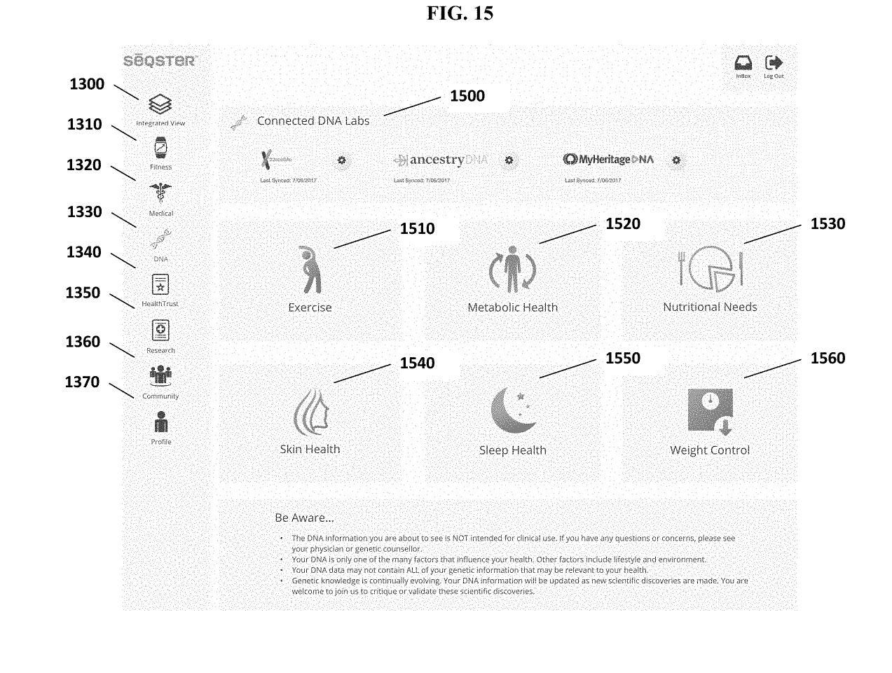

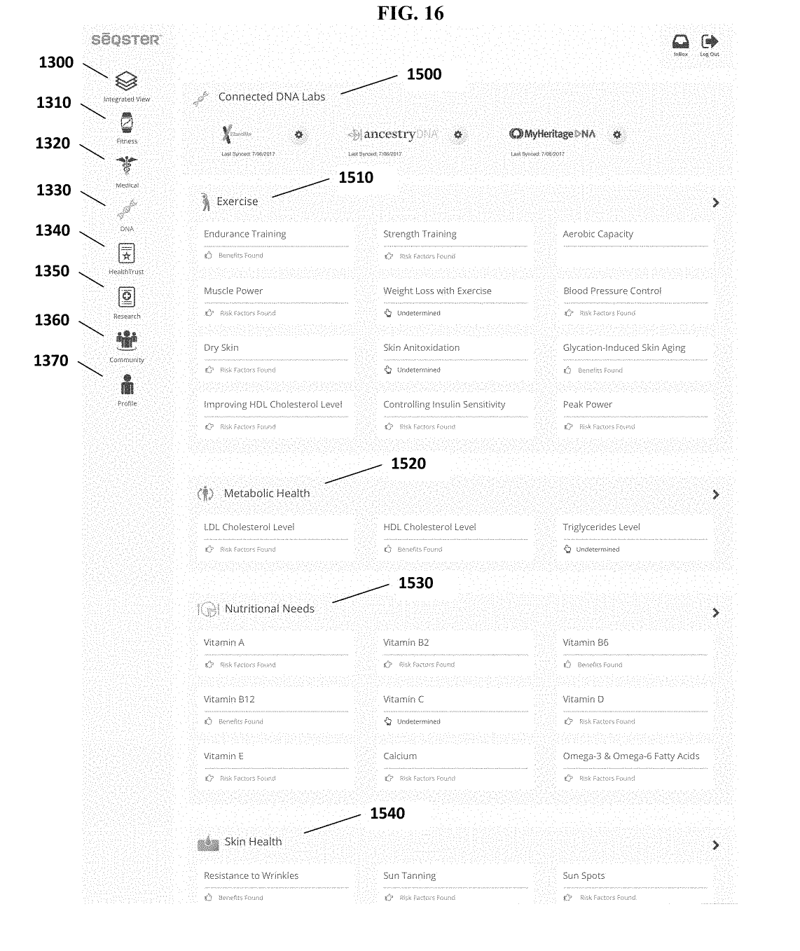

Referring to FIGS. 15 and 16, the health dashboard may include one or more genetic data views. In this example, the health dashboard includes a list of connected genetic sequencing and analysis services 1500. Further, in this example, the health dashboard includes summaries of specific genetic risks, benefits, and/or recommendations such as exercise risks/benefits/recommendations 1510, metabolic risks/benefits/recommendations 1520, nutritional risks/benefits/recommendations 1530, skin risks/benefits/recommendations 1540, sleep risks/benefits/recommendations 1550, and weight risks/benefits/recommendations 1560.

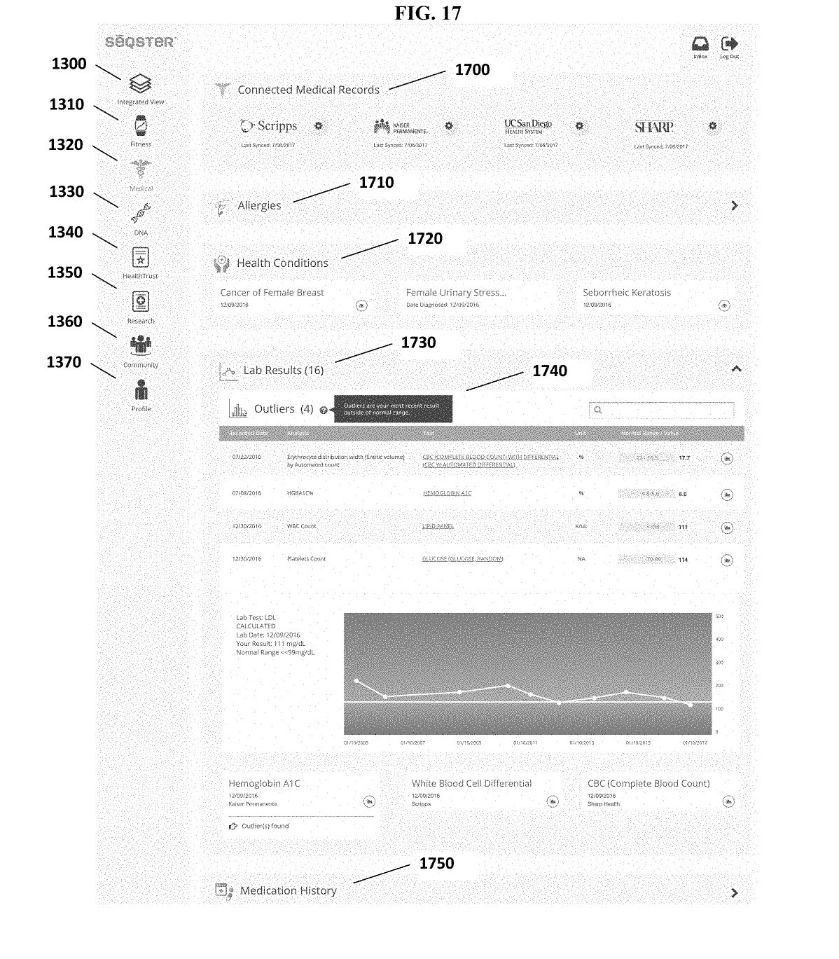

Referring to FIG. 17, the health dashboard may include one or more medical data views. In this example, the health dashboard includes a list of connected EHR providers 1700. Further, in this example, the health dashboard includes summaries of specific relevant health data and events contained in the EHRs such as allergies 1710, health conditions 1720, and lab tests/results 1730. Still further, in this example, the medical data view may include a summary of outlier records 1740 and a summary of medical history 1750.

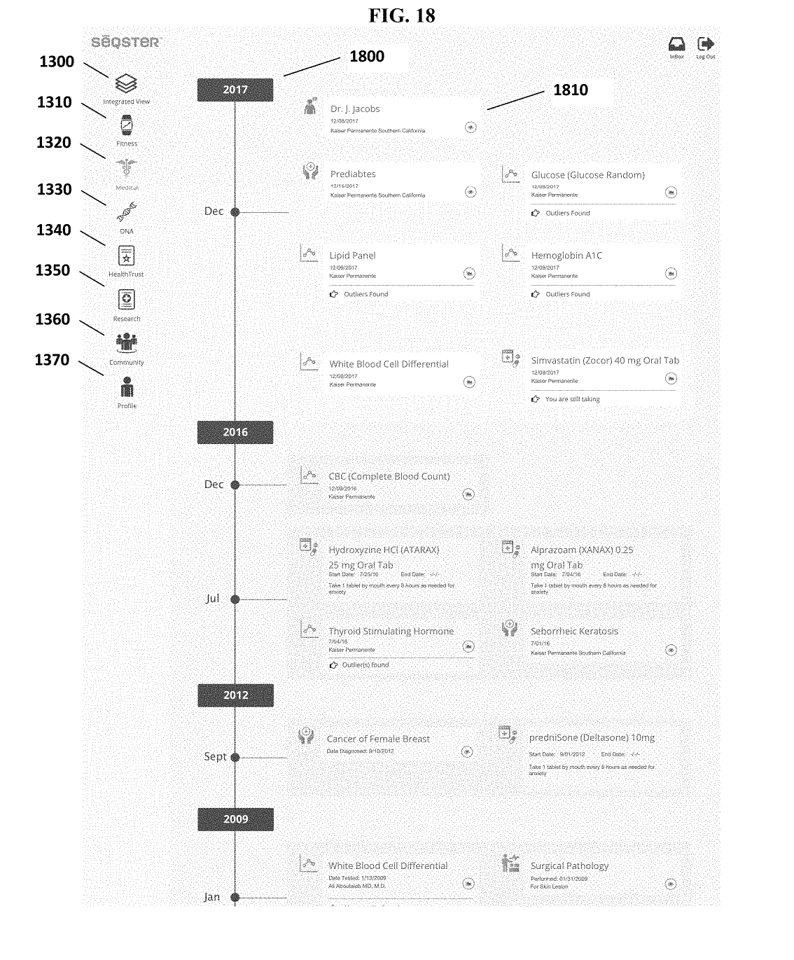

Referring to FIG. 18, the health dashboard may include a timeline view. In this example, the health dashboard includes a timeline 1800 of health-related events 1810. Many health-related events are suitable for display in a chronologically organized format, such as a timeline. Such events may be fitness-related, genetic sequence-related, or medically-related. By way of non-limiting examples, suitable fitness-related events include workouts, calorie burns, changes to trends in fitness data, new fitness data sources, and the like. By way of non-limiting examples, suitable genetic sequence-related events include new variants detected, new research pertaining to a variant, new genetic data sources, and the like. By way of non-limiting examples, suitable medically-related events include appointments, lab tests, lab results, changes to EHRs, new prescriptions, changes to existing prescriptions, diagnoses, therapies, treatments, surgeries, new EHR data sources, and the like. In some cases, the timeline is interactive, allowing a user to change the amount of time depicted, the scale of the timeline, the level of detail, and the like. In further cases, an interactive timeline may allow a user to select, change, or filter the types of health-related events depicted. For example, in some cases, a user may select family members for which health-related events are depicted. By way of further example, a user may select to show or hide one or more of: fitness-related, genetic sequence-related, or medically-related, including sub-categories of the same.

### Health Trust

The platforms, systems, media, and methods described herein include health trusts, or use of the same. An individual creates a health trust by creating their profile, associating one or more types of data, described herein, with their profile, and designating one or more recipient successors for all or part of the profile and/or data to create the health trust. Accordingly, the applications described herein include tools to allow an individual to designate one or more recipient successors for all or part of the profile and the associated data to create a health trust.

In some cases, a health trust protects an individual's profile, and the data associated with the profile, by storing it in an encrypted form and allowing designated recipient successors to gain access to some or all of the profile and/or data in perpetuity. In some cases, a health trust increases the value of an individual's profile, and the data associated with the profile, by increasing the depth and quality of the familial data or by allowing the association of multiple profiles together. By way of example, a health trust allows a parent to provide their children and/or grandchildren with secure and enduring access to their fitness, genetic, and medical data (as well as, optionally, their health directive, will, power of attorney, and estate planning trust). By way of further example, in some cases, when a designated recipient is related to the trustee and becomes a successor to a health trust, if the successor also has a profile or later creates a profile, the successor's profile, and associated data, are linked to, or added to, the health trust to create a deeper and more valuable pool of related data. In such cases, the trust encompasses multigenerational data.

A health trust may be created for some or all of a profile and some or all of the data, described herein, which may be associated with a profile. Moreover, each designated recipient may be named as a successor for some or all of a profile and some or all of the data, described herein, which may be associated with a profile. For example, each designated recipient may be named a successor for one or more of: the fitness data, the genetic data, and the medical data associated with a trustee's profile. By way of further example, each designated recipient may be named a successor for one or more of the individual's health directive, will, power of attorney, and estate planning trust, each of which may, optionally, be associated with the trustee's profile.

In some cases, an individual designates a recipient successor by inputting one or more of: a name, a relationship to the individual, contact information for the recipient successor, and the type or types of data for which the individual should be a successor. When a successor is designated, in some cases, they are sent an invitation informing them of, and allowing them to accept, the designation. In further cases, the invitation includes a link allowing the recipient to accept the invitation. In such cases, once the invitation is accepted the recipient is designated as a successor and has access to the specified part or parts of the profile and the data associated with the profile.

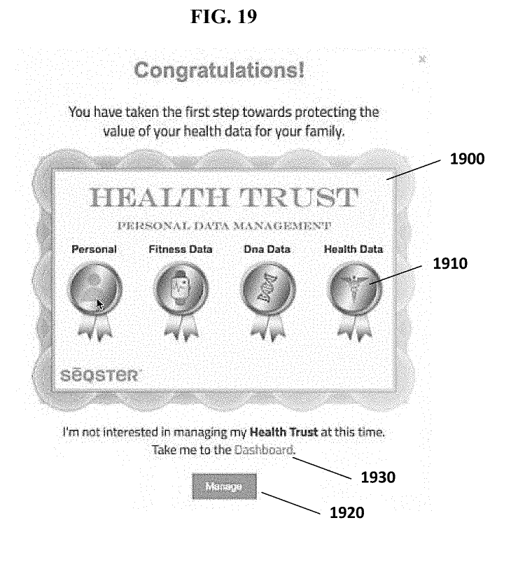

Referring to FIG. 19, a health trust is represented by a health trust certificate 1900. In this example, a health trust certificate 1900 includes a badge 1910 indicating which types of data, described herein, are associated with an individual's profile and therefore included in the health trust. In this case, the health trust certificate 1900 further includes elements allowing the individual to manage their health trust 1920 or to defer management of their health trust 1930 to a later time.

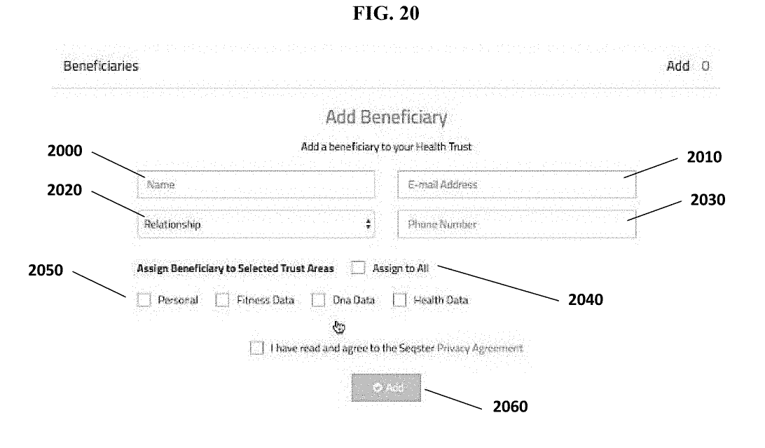

Referring to FIG. 20, an exemplary interface for designating a recipient successor (a beneficiary) includes elements allowing an individual to input the successor's name 2000, email address 2010, relationship to the individual 2020, and phone number 2030. Importantly, the interface for designating a recipient successor further includes elements allowing an individual to specify that the recipient should be designated a successor for all of their profile and associated data 2040 or, alternatively, for one or more specific aspects of, or one or more types of data, such as personal data, fitness data, genetic data, and health data 2050. In this example, interface for designating a recipient successor requires the individual to acknowledge a privacy agreement and allows the individual to further to successor designation process by clicking an “Add” button 2060.

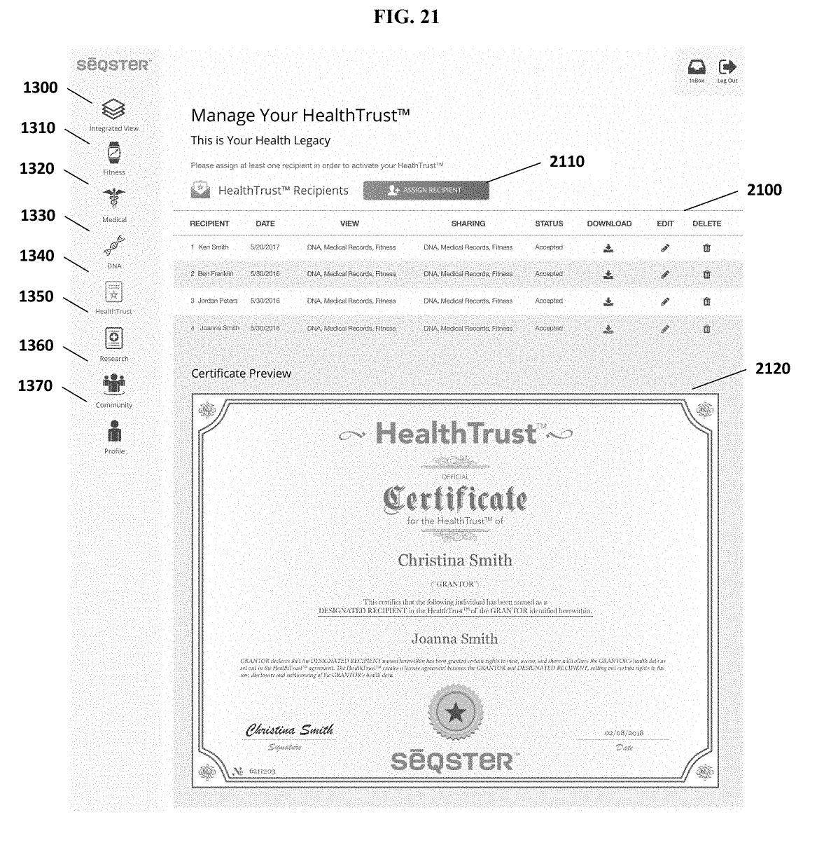

Referring to FIG. 21, the health dashboard may include a health trust management view. In this example, the health trust management view includes a table of current health trust recipients 2100, and for each, a name, date added, sharing status (e.g., list of data types to which they have access), and recipient acceptance status. Also, in this example, the health trust management view includes a GUI element to access tools for adding a recipient to a health trust 2110. Finally, the health trust management view optionally includes a health trust certificate 2120 that may be printed, shared, or transmitted electronically.

### Blockchain Integration

The health trusts described herein benefit from wide availability while maintaining privacy, security, and data and transaction integrity. Blockchain technology may be integrated with the health trust platform, system, and application to facilitate these features. In such cases, the integration of blockchain technology allows a health trust to form an immutable digital trust.

As used herein, a “blockchain” is a continuously growing list of records (e.g., blocks), which are linked and secured using cryptography. Each block may contain a cryptographic hash of the previous block, a date/time stamp, and transaction data. In some cases, a “blockchain,” as described herein, is implemented via a distributed computing network to form a distributed database or distributed ledger.

Blockchain facilitates availability because the technology can provide an open, distributed ledger. For use as a distributed ledger, a blockchain may be managed by a peer-to-peer network. Blockchain facilitates privacy because the technology can use cryptography and cryptographic hashes to protect data. Blockchain facilitates data integrity and transaction integrity because the technology is inherently resistant to modification of the data and can record transactions between two parties in a verifiable and permanent way. By way of example, blockchain technology collectively adheres to a protocol for validating new blocks. Once recorded, the data in any given block cannot be altered retroactively without the alteration of all subsequent blocks, which requires collusion of the network majority. Blockchain also facilitates security because the technology can provide high fault tolerance.

The health trusts described herein optionally use blockchain to track designations, modifications, and/or revocations of recipient successors. The health trusts described herein may use blockchain to track updates to fitness data, genetic data, and/or medical data associated with a trustee's profile. Further, the health trusts described herein may use blockchain to track linked profiles formed by the addition of designated recipient successors' profiles and data to the health trust.

### Algorithms for Generating Value

The platforms, systems, media, and methods described herein include a one or more algorithms for generating a value for a profile, or use of the same. In some cases, the value is a monetary amount associated with access to the profile and its underlying data. The access may be in the form of a data subscription.

The algorithms for generating a value for a profile, and data associated therewith, may utilize one or more of many relevant characteristics of the profile/data when generating a value. By way of examples, the algorithms may utilize, the completeness of the profile, the types of data associated with the profile, the amount of data associated with the profile, the quality of the data associated with the profile, the source(s) of data associated with the profile, how often the data is updated or supplemented, and the like, when generating a value for a profile and data, described herein, that is associated with the profile. By way of further examples, the algorithms may utilize the presence of rare characteristics, such as specific phenotypes and/or genotypes (e.g., rare specific genetic variants), in the data when generating a value for a profile and/or data. By way of still further examples, the algorithms may utilize the number of family members with profiles, the nature of the familial relationships, and depth, breadth, and quality of the profiles of the family members when generating a value for a profile and/or data.

The algorithms may generate a value for a single profile or may generate a value for a group, population, or cohort of profiles. In some cases, the algorithms are weighted algorithms. The algorithms may generate values once or may periodically revise or update the value when the profile or the data associated with the profile changes. In various implementations, the algorithms generate a value for a profile, for example, upon profile creation, yearly, quarterly, monthly, weekly, daily, hourly, upon update to the profile or the associated data, substantially continuously, or in real-time.

A database of individual profiles, each with a machine determined value for access, which is periodically revised as the data is updated, creates a the foundation for a personal data marketplace wherein data consumers identify profiles of interest and remit the determined value in exchange for a subscription to the identified data.

### Profile Search Tools

The platforms, systems, media, and methods described herein include tools for searching the database of profiles, or use of the same. The tools for searching the database of profiles allow data consumers to search, sort, filter, and browse profiles or populations of profiles to identify profiles of interest. In some cases, the tools for searching the database of profiles allow a data consumer to search, sort, or filter based on demographic parameters. In some cases, the tools for searching the database of profiles allow a data consumer to search, sort, or filter based on a specific phenotype. In some cases, the tools for searching the database of profiles allow a data consumer to search, sort, or filter based on a specific genotype, including based on a specific genetic variant such as a SNP. In some cases, the tools for searching the database of profiles allow a data consumer to search, sort, or filter based on a particular medical history, such as prior treatment with a specific therapeutic, a specific outcome (or range of outcomes) of a lab test, or the like. The tools for searching the database of profiles may allow a data consumer to search, sort, or filter based on multiple parameters applied simultaneously. For example, in such cases, a data consumer optionally searches the database of profiles for individuals aged 50+ years, with a diagnosis of type 2 diabetes, treated with metformin, with an A1C level above 7.5 percent, and having SNP rs4402960.

The tools for searching the database of profiles, in some cases, generate search results by use of machine learning algorithms that curate the data and/or identify relevant profiles to present as search results. Such algorithms may generate search results with consideration given to the data consumer's profile, the data consumer's search history, the data consumer's business practices, and the like. The search results are, in some cases, manually curated by a human analyst before or after presentation to a data consumer conducting a search using the profile search tools described herein. In such cases, manual curation serves as a quality assurance process for the search results. Manual curation may also be used as feedback for training machine learning algorithms used to generate the results.

### Recommendation Engine

The platforms, systems, media, and methods described herein include tools for recommending profiles to a particular data consumer, or use of the same. As such, in some cases, the applications described herein include a profile and health data recommendation engine. In some cases, one or more profiles are recommended as relevant to a particular data consumer. Recommendations may be made prior to any search by the data consumer, made along with presentation of results for a search performed by the data consumer, or made as a follow-up to a search performed by the data consumer. The recommendation engine may recommend one profile, a group of profiles (such as related profiles for a family), a cohort of profiles, or a population of profiles.

In some cases, the recommendation engine makes recommendations by use of machine learning algorithms that curate the data and/or identify relevant profiles to present as recommendations. Such algorithms may generate recommendations with consideration given to the data consumer's profile, the data consumer's search history, the data consumer's business practices, and the like. The recommendations are, in some cases, manually curated by a human analyst before or after presentation to a data consumer. In such cases, manual curation serves as a quality assurance process for the recommendations. Manual curation may also be used as feedback for training machine learning algorithms used by the recommendation engine.

### Data Interoperability

The platforms, systems, media, and methods described herein allow individuals integrate their health data into a highly accurate Master Patient Index (MPI) that matches and consolidates patient health data from disparate sources. Health data compatibility, interoperability, and integrity are technical challenges and the platforms, systems, media, and methods described herein include tools for data harmonization, e.g., to facilitate data compatibility and/or interoperability, or use of the same.

For example, the platforms, systems, media, and methods described herein retrieve raw data from many different data sources such as wearable devices, EHR providers, and DNA providers, without the need of going through a long business cycle to get HIPAA business associate agreement (BAA), and meanwhile maintain data integrity and provenance. The described platform ingests and standardizes data from a plurality wearable vendors and fitness app providers, many health providers that are hosted on EHR vendors, including, but not limited to Epic, Cerner, AllScripts, AthenaHealth, and VA (HealtheVet), as well as the raw genome-wide genotyping data provided by direct-to-consumer DNA labs including, by way of examples, 23andMe, Ancestry.com, MyHeritage, and FamilyTreeDNA.

In some cases, methodologies are utilized to harmonize raw wearable, EHR, and DNA data across different sources as a first step towards addressing the challenge of health data interoperability. In brief, raw data may go through three stages of transformation. First, in some cases, raw data are parsed according to its source type (e.g., HL7 v2, CCDA, Custom API, etc.) and transformed into JavaScript Object Notation (JSON) format. Secondly, in further cases, the JSON formatted data is stored in structured relational database tables and become easily searchable entities. Finally, in still further cases, the structured data across multiple sources are standardized into a consistent set of terms that allow users to see longitudinal data from fitness activities, biometrics, lab results, and medication usage across disparate sources. This process unifies various coding systems like ICD-9/10, RxNorm, and LOINC into a health data interoperability framework.

In addition, the subject matter described herein employs intuitive and communicative charts and dashboards so that individuals can make sense of their voluminous and complex health data. These views (see, e.g., FIGS. 13-18 and 21) are essential to maintain engagement of individuals in their own health management. With matched genetic, medical, and fitness data aggregated in one place, the platforms, systems, media, and methods described herein include the unique capability to visualize data in an integrated fashion for users, empowering them to comprehend their health in a broad context not otherwise possible and enabling the identification of novel health factor correlations and discover emerging trends.

The highly fragmented state of U.S. healthcare system imposes an additional technical challenge. No data standards have been adopted for patient portals, thus each provider may implement their patient portal in a different manner. Flexible software modules to interface with and ingest data from these patient portals are required. To improve computational efficiency, the subject matter described herein utilizes a proprietary EHR retriever suite that can automatically identify the EHR vendor with high accuracy and then retrieve medical data by predicting possible API call endpoints learned over time about the particular EHR vendor. This EHR retriever suite may optionally be trained on many top EHR vendors such as Epic and Cerner.

A large proportion of the codebase described herein is dedicated to techniques to standardize disparate health data sources across multiple providers into a consistent set of terms that allow users to see longitudinal data from fitness activities, biometrics, lab results, and medication usage independent of the data sources. However, even with these techniques in place, EHR data standardization remains a significant challenge particularly when medical terms used in EHRs are not linked to medical codes as described above. Instead of preemptively standardizing all possible non-coded medical terms, which is an enormous task requiring an immeasurable amount of time and effort, the platforms, systems, media, and methods described herein, in some cases, utilize a proprietary ontology mapping and guided curation tool that enables its medical staff to standardize any medical term into the health data interoperability framework as novel terms are observed by the system.

### Data Subscriptions and Licenses

The platforms, systems, media, and methods described herein include tools for subscribing to and/or licensing profile data, or use of the same. A data subscription may be directed to a single profile, or a group, population, or cohort of profiles, such as a family group. In some cases, a data subscription allows a data consumer, having paid for the subscription, to access one or more profiles and data, described herein, associated with those profiles. In further cases, a subscriber is notified when the profile or its associated data is updated, revised, or modified and provided with access to the updated, revised, or modified data. A data consumer subscribing to a profile may, in some cases, be enabled to communicate with the individual owner of the profile and/or may be enabled to extend offers to the individual.

Similarly, a data license may be directed to a single profile, or a group, population, or cohort of profiles, such as a family group. In some cases, a data license allows a data consumer, having paid for the license, to access and/or use one or more profiles and data, described herein, associated with those profiles. In further cases, a licensee is notified when the profile or its associated data is updated, revised, or modified and provided with access to, and optionally the right to use, the updated, revised, or modified data. A data consumer licensing a profile may, in some cases, be enabled to communicate with the individual owner of the profile and/or may be enabled to extend offers to the individual.

The data subscriptions and/or data licenses may be implemented via blockchain technology to create a record of data consumers subscribing to and/or licensing a particular profile or to create a record of profiles subscribed to and/or licenses by a particular data consumer. For example, data subscriptions and licenses may be recorded in a distributed database that maintains a continuously growing list of ordered records (e.g., blocks), wherein each block contains a timestamp and a link to a previous block. Such records allow the individual owner of a profile to manage subscriptions, licenses, and audit revenue obtained from subscribers/licensees for their profile/data and those for which they may be a designated recipient successor.

In some cases, a data subscription is obtained for a one-time fee. In other cases, a data subscription is obtained on the basis of an ongoing, periodic fee. In further cases, at least a part of the fees are provided to the owner or owners of the profiles to which the data consumer has subscribed. Similarly, a data license may be obtained for a one-time fee or, alternatively, obtained on the basis of an ongoing, periodic fee. A data license described herein may be exclusive or non-exclusive based on the preferences of the individual and/or the data consumer. In further cases, at least a part of the fees are provided to the owner or owners of the profiles licensed by the data consumer.

### Architecture

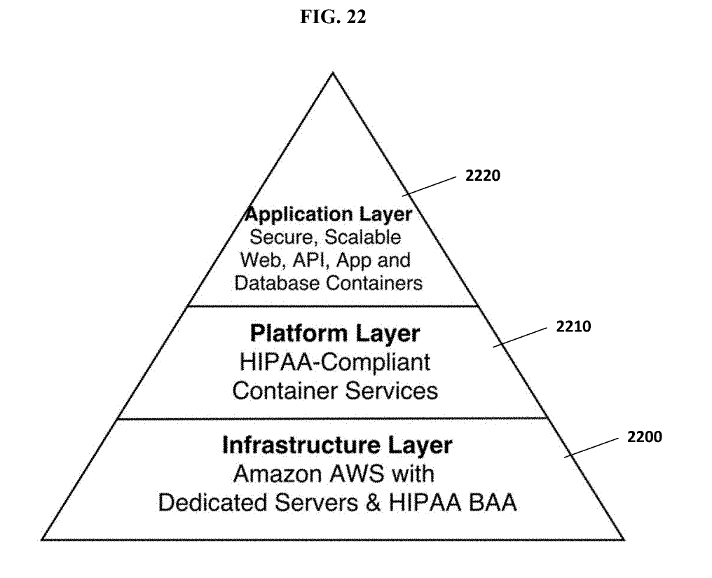

A non-limiting, exemplary, but suitable system architecture is illustrated in FIG. 22. This example illustrates a system designed with a multi-layer architecture designed for scalability and security. At the infrastructure layer 2200, the system is hosted on Amazon AWS with a business associate agreement (BAA) in place for managing personal health information (PHI) and de-identified clinical data on dedicated servers in a virtual private cloud (VPC).

Further, in this example, above the AWS infrastructure is a highly scalable platform layer 2210 comprising container services architecture which uses Docker containers within a secure PaaS (Platform as a Service) environment that is monitored 24×7×365 by a vendor that specializes in hosting HIPAA, PHI and PII-compliant data for companies that range from startups to university health systems, to pharmaceutical companies. The vendor is HIPAA and ISO-27001 compliant and provides a 99.95% uptime service level agreement and a HIPAA Breach Indemnification Agreement.

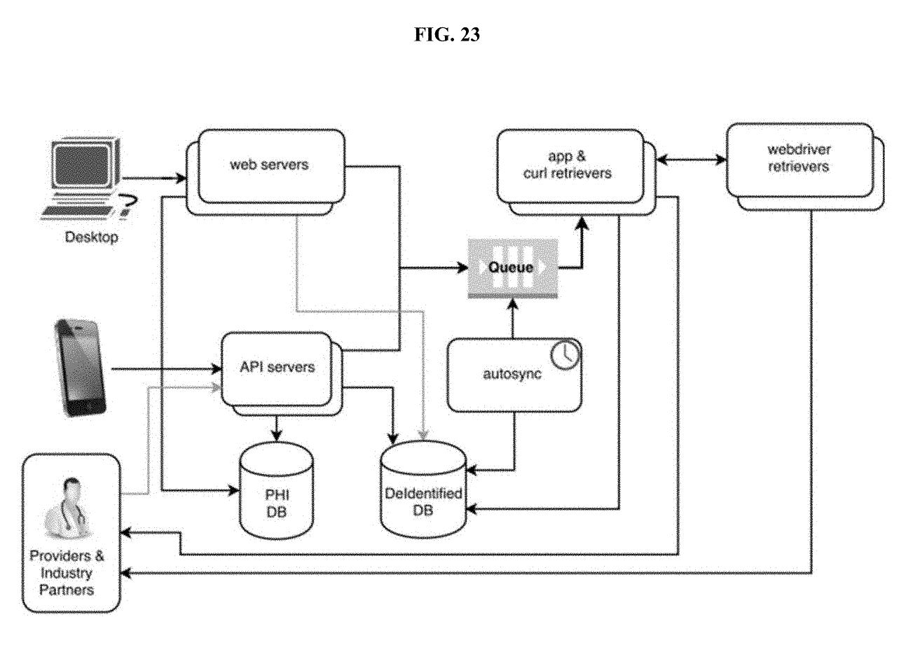

A non-limiting, exemplary, but suitable application layer 2220 architecture is illustrated in FIG. 23. This example illustrates a system that, at the application layer, only exposes the minimum footprint of web containers endpoints for web and mobile edge traffic from consumers as well as health, fitness and lab partners that need API access to Seqster backend data.

In the examples provided herein, the web and API servers auto-scale on demand. As requests come in, any task that takes more than, for example, 1-2 seconds to return data is dispatched to a job queue. A dynamically scalable set of app servers and retriever servers are allocated based on demand and then scaled back down automatically during idle periods. In some cases, the systems described herein comprise two types of retrievers: 1) fast “curl” retrievers which can quickly connect to APIs and portals using http and retrieve data in seconds; and 2) “webdriver” retrievers which retrieve data that is not directly accessible via API but which can be retrieved by browser activity simulation. In such cases, retrievers cover all facets of digital health data including fitness, nutrition, biometrics, electronic health records, and DNA sequence data.

In some cases, one or more fitness and/or EHR providers push data to the described API servers either shortly after an activity has completed or at regular intervals such as once a day. For those providers that do not push data to the API servers, a robust auto-sync subsystem may retrieve data at per-provider intervals (for example, daily for fitness and weekly for EHR).

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, GoogleTV®, Google Chromecast®, Amazon Fire®, and Samsung® HomeSync®. Those of skill in the art will also recognize that suitable video game console operating systems include, by way of non-limiting examples, Sony® P53®, Sony® P54®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

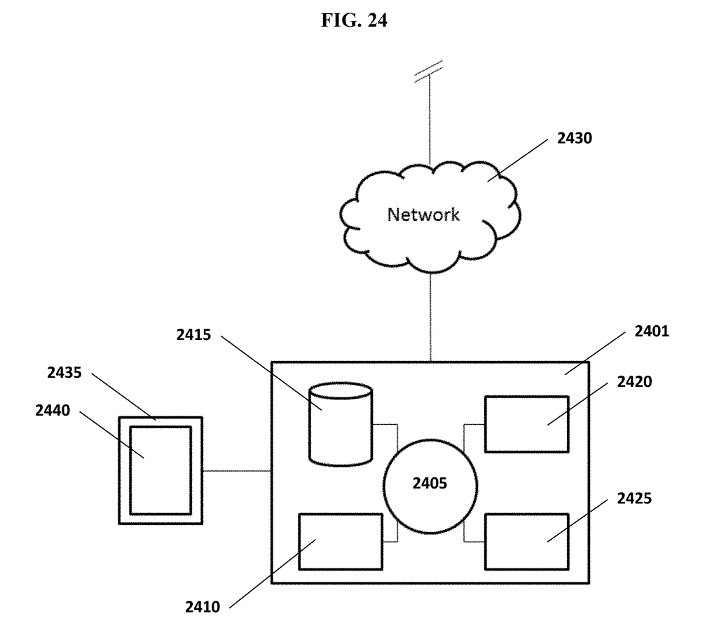

Referring to FIG. 24, in a particular embodiment, an exemplary digital processing device 1501 is programmed or otherwise configured to store profiles, ingest health data from external sources, value individual profiles, and/or provide interfaces for searching profiles. The device 2401 can regulate various aspects of application of the valuation algorithms and/or maintenance of the blockchain technology of the present disclosure. In this embodiment, the digital processing device 2401 includes a central processing unit (CPU, also “processor” and “computer processor” herein) 2405, which can be a single core or multi core processor, or a plurality of processors for parallel processing. The digital processing device 2401 also includes memory or memory location 2410 (e.g., random-access memory, read-only memory, flash memory), electronic storage unit 2415 (e.g., hard disk), communication interface 2420 (e.g., network adapter) for communicating with one or more other systems, and peripheral devices 2425, such as cache, other memory, data storage and/or electronic display adapters. The memory 2410, storage unit 2415, interface 2420 and peripheral devices 2425 are in communication with the CPU 2405 through a communication bus (solid lines), such as a motherboard. The storage unit 2415 can be a data storage unit (or data repository) for storing data. The digital processing device 2401 can be operatively coupled to a computer network (“network”) 2430 with the aid of the communication interface 2420. The network 2430 can be the Internet, an internet and/or extranet, or an intranet and/or extranet that is in communication with the Internet. The network 2430 in some cases is a telecommunication and/or data network. The network 1530 can include one or more computer servers, which can enable distributed computing, such as cloud computing. The network 2430, in some cases with the aid of the device 2401, can implement a peer-to-peer network, which may enable devices coupled to the device 2401 to behave as a client or a server.

Continuing to refer to FIG. 24, the CPU 2405 can execute a sequence of machine-readable instructions, which can be embodied in a program or software. The instructions may be stored in a memory location, such as the memory 2410. The instructions can be directed to the CPU 2405, which can subsequently program or otherwise configure the CPU 2405 to implement methods of the present disclosure. Examples of operations performed by the CPU 2405 can include fetch, decode, execute, and write back. The CPU 2405 can be part of a circuit, such as an integrated circuit. One or more other components of the device 2401 can be included in the circuit. In some cases, the circuit is an application specific integrated circuit (ASIC) or a field programmable gate array (FPGA).

Continuing to refer to FIG. 24, the storage unit 2415 can store files, such as drivers, libraries and saved programs. The storage unit 2415 can store user data, e.g., user preferences and user programs. The digital processing device 2401 in some cases can include one or more additional data storage units that are external, such as located on a remote server that is in communication through an intranet or the Internet.

Continuing to refer to FIG. 24, the digital processing device 2401 can communicate with one or more remote computer systems through the network 2430. For instance, the device 2401 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the digital processing device 2401, such as, for example, on the memory 2410 or electronic storage unit 2415. The machine executable or machine readable code can be provided in the form of software. During use, the code can be executed by the processor 2405. In some cases, the code can be retrieved from the storage unit 2415 and stored on the memory 2410 for ready access by the processor 2405. In some situations, the electronic storage unit 2415 can be precluded, and machine-executable instructions are stored on memory 2410.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

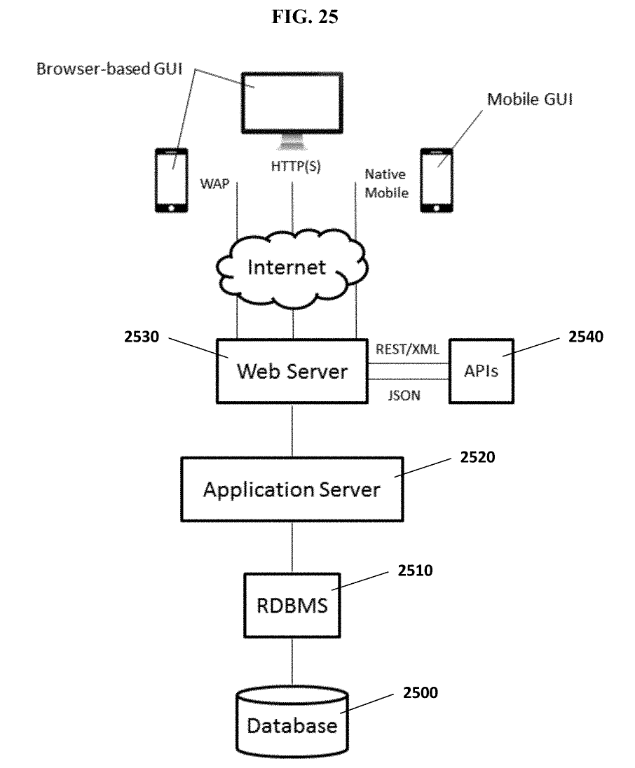

Referring to FIG. 25, in a particular embodiment, an application provision system comprises one or more databases 2500 accessed by a relational database management system (RDBMS) 2510. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 2520 (such as Java servers, .NET servers, PHP servers, and the like) and one or more web servers 2530 (such as Apache, IIS, GWS and the like). The web server(s) optionally expose one or more web services via app application programming interfaces (APIs) 2540. Via a network, such as the Internet, the system provides browser-based and/or mobile native user interfaces.

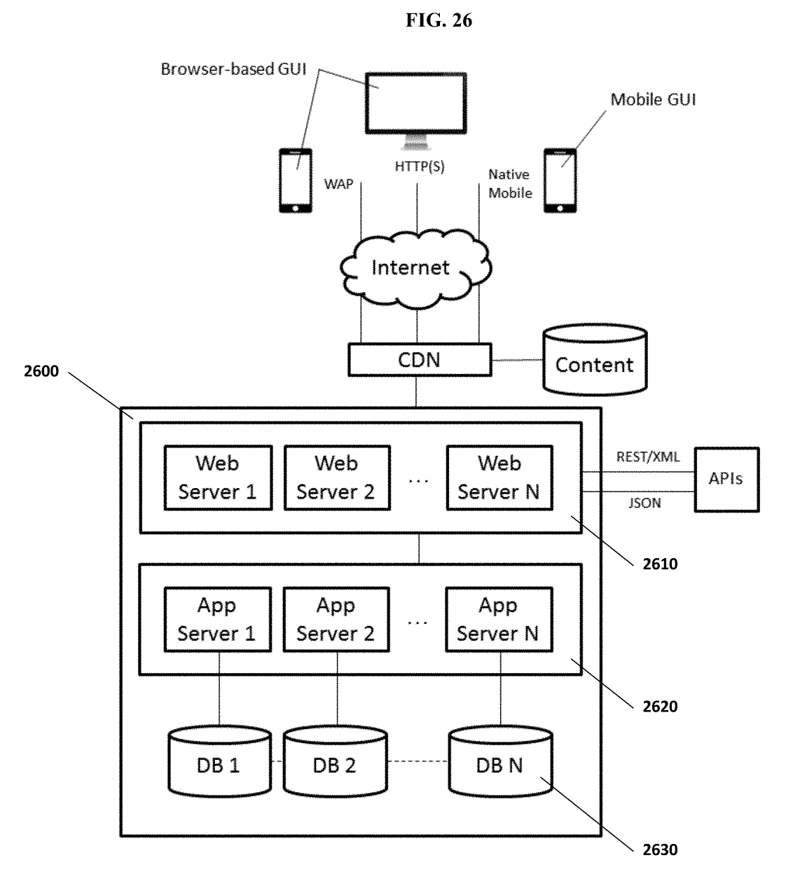

Referring to FIG. 26, in a particular embodiment, an application provision system alternatively has a distributed, cloud-based architecture 2600 and comprises elastically load balanced, auto-scaling web server resources 2610 and application server resources 2620 as well synchronously replicated databases 2630.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of profile, fitness, genetic, health, profile value, and trust information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

1. A computer-implemented method of managing health information:
a) providing tools for an individual to create a profile, the profile comprising personal information;
b) ingesting fitness data, genetic data, and medical data for the individual and associating the fitness data, the genetic data, and the medical data with the profile; and
c) providing tools for the individual to designate one or more recipient successors for all or part of the profile and the associated data to create a health trust.

2. The method of claim 1, further comprising harmonizing the ingested data through a staged transformation process while maintaining integrity and provenance of the data, the staged transformation process comprising:
a) parsing the ingested data according to source type; and
b) standardizing the parsed data into a consistent set of terms;
thereby forming a health data interoperability framework.

3. The method of claim 2, further comprising formatting the data into JavaScript Object Notation (JSON) format and storing the formatted data in structured relational database tables.

4. The method of claim 2, further comprising providing an ontology mapping and guided curation tool to enable medical staff to standardize any medical term into the health data interoperability framework.

5. The method of claim 1, wherein the individual designates each recipient as a successor for one or more of: the personal information, the fitness data, the genetic data, and the medical data.

6. The method of claim 1, wherein the individual designates each recipient successor by inputting a name, a relationship to the individual, and contact information for the recipient successor.

7. The method of claim 1, wherein the method further comprises sending an invitation to each designated recipient successor, the invitation comprising a feature allowing the recipient to accept the invitation.

8. The method of claim 7, wherein the one or more recipient successors accepting the invitation have access to all or part of the profile and the associated data.

9. The method of claim 1, wherein the personal information comprises social networking information.

10. The method of claim 1, wherein the personal information comprises family tree information.

11. The method of claim 1, wherein the fitness data comprises data generated by a fitness tracking device or input by the user into a fitness log.

12. The method of claim 11, wherein the fitness data is ingested by accessing an API or by receiving upload of one or more data files provided by the individual.

13. The method of claim 1, wherein the genetic data comprises nucleic acid sequence information.

14. The method of claim 13, wherein the nucleic acid sequence information comprises DNA sequence information or RNA sequence information.

15. The method of claim 13, wherein the genetic data is ingested by accessing an API or by receiving upload of one or more data files provided by the individual.

16. The method of claim 1, wherein the medical data comprises at least one electronic health record (EHR) or at least one personal health record (PHR).

17. The method of claim 16, wherein the medical data is ingested by accessing an API or by receiving upload of one or more data files provided by the individual.

18. The method of claim 1, wherein the method further comprises providing a coupon, discount, or subsidy for genetic sequencing services in response to the ingesting of the fitness data, the medical data, or both the fitness data and the medical data.

19. The method of claim 1, wherein the method further comprises generating and presenting a genetic data summary for the individual comprising a plurality of phenotypes, the genetic data summary further comprising at least one annotation, at least one article, and a confidence level associated with each phenotype.

20. The method of claim 1, wherein the method further comprises applying an algorithm to generate a monetary market value for the profile and the associated data, and wherein the monetary market value is based, at least in part, on one or more of: the quantity of the data, the quality of the data, the number of types of data, the number of family members of the individual who have profiles, and whether a health trust is established.

21-35. (canceled)
